(19) United States
(12) Patent Application Publication
(10) Pub. No.: US 2015/0126392 A1
(43) Pub. Date: May 7, 2015

(54) METHOD, SYSTEM, AND KIT FOR CHARACTERIZING A CANCER

(71) Applicant: Vanderbilt University, Nashville, TN (US)

(72) Inventors: Yajun Andrew Yi, Nashville, TN (US); Alfred L. George, JR., Nashville, TN (US)

(21) Appl. No.: 14/531,869

(22) Filed: Nov. 3, 2014

Related U.S. Application Data
(60) Provisional application No. 61/898,989, filed on Nov. 1, 2013.

(51) Int. Cl. C12Q 1/68

(52) U.S. Cl. CPC C12Q 1/6886; C12Q 2600/158; C12Q 2600/118
USPC 506/9; 506/16

(57) ABSTRACT

A method for characterizing a cancer in a subject, comprising:
(a) providing a biological sample from the subject;
(b) determining expression levels of genes in the biological sample to obtain a gene expression profile for the subject, wherein the genes comprise members of a meta-signature associated with the cancer; and
(c) comparing the profile of the subject to a reference, wherein the cancer is characterized based on a measurable difference in the expression levels of genes in the biological sample as compared to the reference.

### RELATED APPLICATIONS

This application claims priority from U.S. Provisional Application Ser. No. 61/898,989 filed Nov. 1, 2013, the entire disclosure of which is incorporated herein by this reference.

### GOVERNMENT INTEREST

This invention was made with government support under CA114033 awarded by National Cancer Institute (NCI). The government has certain rights in the invention.

### TECHNICAL FIELD

The presently disclosed subject matter relates to methods for characterization of and evaluation of treatment and/or progression of a cancer. In particular, the presently-disclosed subject matter relates to methods involving a determination of expression levels of genes in a biological sample from a subject to obtain a gene expression profile for the subject, where the genes include members of a meta-signature associated with the cancer.

### BRIEF DESCRIPTION OF THE DRAWINGS

FIG. 1. Signature clustering process for identification of BRmet50. The workflow of iterative EXALT method includes three major processes. (1) Extraction of 633 breast cancer signatures. All paired sample groups within each breast cancer datasets (n=223) were compared based on all possible clinical and pathologic covariates such as tumor size, nodal involvement, grade, marker status, lymphovascular invasion, relapse, metastasis, p53 status, BRCA1 and BRCA2 mutations. Student's t-test was then performed for all pairwise comparisons, and a total of 633 breast cancer signatures were generated and uploaded into a database (HuCaSigDB). (2) Signature clusters and classification. Iterative search was carried out using each of 633 signatures as a query (anchored or seed) signature against HuCaSigDB repeatedly to identify homologous signatures with significant data similarity defined by EXALT. 121 out of 633 query signatures found at least one similar signature in HuCaSigDB and formed 121 clusters, while the remaining 512 (singletons) failed to generate clusters. Two typical results are depicted by schematic description labeled with anchored signatures: the singleton Sig21 and the cluster Sig24 including 11 signature members like Sig544, Sig128, Sig140, etc. Knowledge based analysis of signature phenotypes and sizes was performed among 121 signature clusters. Eight clusters had obvious metastasis phenotypes. Of the eight clusters, the largest cluster anchored by the query signature (sig24) was selected for further analysis. (3) Identification of meta-signature BRmet50. All 6,526 signature genes from the 11 signatures of the cluster Sig24 were assembled together to form a synthetic signature (BRmet). The genes within BRmet were ranked based on recurrent frequency and concordance of differential expression represented by a meta-heat map. The top 50 genes (BRmet50) represented in rows were determined by a 100% recurrent frequency and gene expression profile concordance among the 11 signatures represented in columns. The colors in the meta-heat map represent the direction of differential gene expression within a given transcriptional profile (red for up, green for down, and black for a missing match). Color intensity reflects the confidence levels of differential expression.

FIG. 2. Kaplan-Meier analyses for relapse-free survival. Data from 108 tumors from the dataset BR1042 were stratified into two groups by BRsig70 and BRsig76 (bottom panels), the control signature (BRmet[-1042]) from the leave-one-out method, or BRmet50 (upper panels) gene expression profiles. In each survival plot, two types of relapse-free survival were compared: a poor prognosis group (black dashed line) and a good prognosis group (red solid line). The relapse-free time in days is displayed on the x-axis, and the y-axis shows the probability of relapse-free survival. The p-values indicate the statistical significance of survival time differences between the two groups.

Figure S1. Flow chart of statistical methods for validation. Four types of signatures were used in this study: (1) BRmet50; (2) BRmet50 control signatures from “Leave-one-out” process; (3) BRsig70, BRsig76 and other six known signatures for breast cancer prognosis; and (4) 1,000 random signatures of identical in size to BRmet50. Gene expression signatures were used for unsupervised hierarchical clustering. Sample group assignments were determined in each data set based on the sample clustering dendrogram. Gene expression-based sample groups together with patient survival data and clinicopathological variables in cancer were used to determine the signature prognostic performance in survival analyses. The survival analyses include log-rank tests and Cox proportional hazards regression models (univariate and multivariate models). All signatures were validated in 21 breast cancer (BR) data sets (Table 2, Supplemental Figures S2 and 2 and Supplemental Table S7), and one breast cancer data set (BR1141) was further analyzed among breast cancer subsets (Table 3 and Supplemental Table S4). BRmet50, BRsig70, and BRsig76 were examined to determine whether they were independent of common clinicopathologic factors in breast cancer (Table 4, Supplemental Tables S5, S6) and three other three cancer types (Table 5).

Figure S2. Comparison of cancer signatures and random signatures.

A total of 21 datasets were tested individually with 1,000 random signatures and seven known cancer signatures. Each panel is labeled with its respective test dataset ID and depicts the distribution of p-values from 1,000 random signatures identical in size to BRmet50 (50 genes). The x-axis denotes the reciprocal logarithm of p-value (-log [p-value]) from survival analyses. Colored arrowheads represent the seven known cancer signatures and point to the p-value locations in the random p-value distributions.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods and kits for characterizing a cancer in a subject, and for evaluating treatment efficacy and/or progression of a cancer in a subject.

In some embodiments, a method for characterizing a cancer in a subject is provided and includes providing a biological sample from the subject; determining expression levels of genes in the biological sample to obtain a gene expression profile for the subject, wherein the genes comprise members of a meta-signature associated with the cancer; and comparing the profile of the subject to a reference, wherein the cancer is characterized based on a measurable difference in the expression levels of genes in the biological sample as compared to the reference. In some embodiments, the characterizing comprises providing a diagnosis, prognosis and/or theranosis of the cancer. In some embodiments, the method can include applying an algorithm for predicting a clinical outcome indicator from the gene expression profile of the subject, including genes comprising members of the meta-signature.

In some embodiments, a method for evaluating treatment efficacy and/or progression of a cancer in a subject is provided and includes providing a biological sample from the subject; determining expression levels of genes in the biological sample to obtain a gene expression profile for the subject, wherein the genes comprise members of a meta-signature associated with the cancer; and comparing the profile of the subject to a reference, wherein the treatment efficacy and/or progression of the cancer is evaluated based on a measurable difference in the expression levels of genes in the biological sample as compared to the reference. In some embodiments, the method also involves providing multiple biological samples from the subject collected at different time points, and determining expression levels of genes in each biological sample.

As used herein, cancer can refer to a breast cancer, a lung cancer, a prostate cancer, and a colon cancer.

In certain instances, nucleotides and polypeptides associated with particular genes disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this application.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### Examples

The present inventors implemented and performed a large meta-analysis of breast cancer gene expression profiles from 223 datasets containing 10,581 human breast cancer samples using a novel data similarity-based approach (iterative EXALT). Cancer gene expression signatures extracted from individual datasets were clustered by data similarity and consolidated into a meta-signature with a recurrent and concordant gene expression pattern. A retrospective survival analysis was performed to evaluate the predictive power of a novel meta-signature deduced from transcriptional profiling studies of human breast cancer. Validation cohorts consisting of 6,011 breast cancer patients from 21 different breast cancer datasets and 1,110 patients with other malignancies (lung and prostate cancer) were used to test the robustness of our findings. During the iterative EXALT analysis, 633 signatures were grouped by their data similarity and formed 121 signature clusters. From the 121 signature clusters, we identified a unique meta-signature (BRmet50) based on a cluster of 11 signatures sharing a phenotype related to highly aggressive breast cancer. In patients with breast cancer, there was a significant association between BRmet50 and disease outcome, and the prognostic power of BRmet50 was independent of common clinical and pathologic covariates. Furthermore, the prognostic value of BRmet50 was not specific to breast cancer, as it also predicted survival in prostate and lung cancers.

Conclusions: We have established and implemented a novel data similarity-driven meta-analysis strategy. Using this approach, we identified a transcriptional meta-signature (BRmet50) in breast cancer, and the prognostic performance of BRmet50 was robust and applicable across a wide range of cancer-patient populations.

Introduction

Breast cancer is the most common type of cancer in women and the second-leading cause of cancer death among women in the United States. A molecular biomarker that can predict the likelihood of cancer progression to invasive or metastatic disease can guide how aggressively patients are initially treated [1]. There is a clear need for a better understanding of how molecular profiles relate to cancer phenotypes and clinical outcomes and for new cancer biomarkers with definable and reproducible performance in diverse patient populations.

The introduction of genome-scale gene expression profiling has led to the identification of specific transcriptional biomarkers known as gene expression signatures. The discovery of gene expression signatures from any single well-powered study is relatively straightforward. Some signatures have utility as transcriptional biomarkers for classifying patients with significantly different survival outcomes in breast cancer [2,3]. For example, transcriptional profiling of primary breast cancer has been used previously to identify a 70-gene signature (marketed as MammaPrint but designated here as BRsig70) [3], a distinct 76-gene signature (BRsig76) [2], and others (Oncotype DX [4,5], TAMR13 [6], Genius [7], GGI [8], PAM50 [9] and PIK3CAGS278 [10]). Typical of other transcriptional biomarkers, both BRsig70 and BRsig76 were derived from a training set from a single study and then validated with a test set from the same retrospective patient cohorts. When subjected to external validation, most signatures could only be validated using one dataset (NKI295) [11] or a few smaller datasets with retrospectively accrued samples. This validation method has inevitable limitations of statistical power or sample selection bias. As a result, a common weakness of this approach is its lack of consistency and reproducibility [12-16].

With hundreds of breast cancer gene expression datasets deposited in public databases, we now have the ability to utilize these data to their full potential and discover recurrent and reliable gene expression signatures for breast cancer prognosis prediction. However, the identification of a prognostic expression signature through meta-analysis of publicly available cancer gene expression profiles represents an underexploited opportunity. There are several reports of meta-analysis frameworks that use multiple breast cancer datasets to build and validate prognostic classifiers [7,17,18]. These approaches focus on selecting predictors from combined training sets, either using average Cox-scores [18] or taking into account the sample molecular subtypes [7,17]. However, one unanswered question is how to identify homogeneous gene expression studies using a refined and unbiased selection method [19]. In order to extrapolate validated prognostic signatures to a broader patient population, new biostatistical methods using data similarity-based analysis are needed [20].

To avoid the weaknesses of single study-derived signatures and to generate a new strategy to better utilize the available gene expression data from independent studies, we have developed a meta-analysis strategy called EXALT (EXpression AnaLysis Tool) [21,22]. The essential feature of EXALT is a database containing thousands of gene expression signatures extracted from published studies that enables signature comparisons. In this study, we used EXALT in an iterative manner (iterative EXALT) to conduct a data similarity-driven meta-analysis and elucidate transcriptional signatures with enhanced prognostic value in breast cancer. We demonstrated that heterogeneous signatures from 223 public datasets containing 10,581 breast cancer samples could be systematically organized by their common data elements (i.e., intrinsic similarities and disease phenotypes) and assembled into a new signature data type called a meta-signature. We identified a specific meta-signature consisting of 50 genes (BRmet50) that is robustly predictive of cancer prognosis in 6,011 breast cancer patients from 21 different breast cancer datasets as well as in other malignancies including lung and prostate cancer. These findings illustrate the value of BRmet50 in breast cancer prognosis independent of treatment variables and indicate that iterative EXALT is a novel meta-analysis method capable of performing informative and robust discovery of meta-signatures in cancer.

Methods

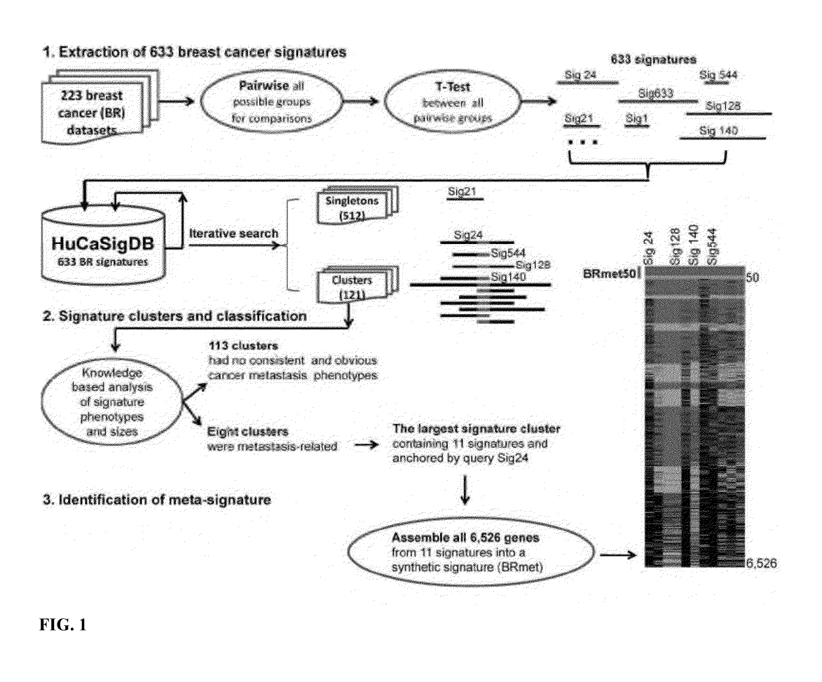

The methods used for signature extraction, developing the signature databases, and EXALT analysis were previously reported [21,22]. Iterative EXALT analysis for clustering and assembling signatures is described in the result section (FIG. 1) and the Supplemental Methods.

Patient Data

Patient information, both clinical data and gene expression data for signature identification and validation, were obtained from independently published human cancer studies and the Gene Expression Omnibus (GEO) provided by the National Center for Biotechnology Information (NCBI) [23] as described in Supplemental Table S1, Table 1, Table 2, and Table 5. The meta-signature (BRmet50) was derived from meta-analysis of breast cancer gene expression profiles from 223 breast cancer training datasets (Supplemental Table S1). Leave-one-out cross validation was used to prepare BRmet50 control signatures from nine training datasets (Supplemental Table S2) as described in the Supplemental Methods. To provide an evaluation of the iterative EXALT approach and meta-signature, 21 datasets (Table 2) containing 6,011 breast tumor samples were retrospectively examined by survival analyses [2,3,6,8,11,24-36]. Of them, 10 are from 223 training datasets (Table 1 and Table 2), and the other 11 (“validation datasets” in Table 2) are independent validation datasets not included in the 223 training datasets (Supplemental Table S1).

To ensure quality in the test survival data sets derived from published breast cancer studies, we applied the “rule of fifty” [37-39] as an inclusion criteria. Specifically, an included dataset must have at least 50 samples with survival data (designated as survival samples) and a minimum of 10 events. To ensure a valid sample size for a survival analysis, at least 60% of the samples were required to have survival information. Thus, missing data (censored survival data) was controlled to a minimal level. The average follow-up length was 14 years across 21 datasets.

Statistical Analysis

Our statistical approach, as illustrated in Supplemental Figure S1, assessed the ability of the identified meta-signature BRmet50 to serve as a survival time predictor. First, hierarchical clustering of the BRmet50 gene profiles in each test dataset was performed and visualized using the open-source desktop program (version 1.5.0.Gbeta) developed at Vanderbilt University. Spearman rank correlation was used to measure the similarities in gene expression profiles among patient samples. Unsupervised hierarchical clustering based on average linkage was performed to group the patient samples. The group assignments for the patient samples were determined in each dataset based on the first bifurcation of the clustering sample dendrogram [40]. Using disease outcomes, Kaplan-Meier curves for the two groups were compared. Log-rank tests and c-index measurements were conducted for the two groups' survival difference. The Cox proportional hazards model was applied to each dataset for both univariate and multivariate survival analyses. All these analyses were carried out with the open-source R software, version 2.14.1 (www.r-project.org).

For general prognosis performance evaluation of various signatures in full datasets and subsets, p-values from log-rank tests and from univariate and multivariate Cox proportional hazard models were evaluated. Various disease outcomes (e.g., relapse, distant metastasis, or death) were used as clinical end points (Table 2). The estimated hazard ratio (HR), its 95% confidence interval (CI), and the p-value allowed us to directly compare the performances of different signatures (Supplemental Figure S1). For graphical representation, Kaplan-Meier curves of survival probability were plotted for each subgroup.

Results

Extraction of Human Cancer Signatures

To organize the complex transcriptional data, we have established a hierarchical data structure. The top level consists of the transcriptional studies, and each transcriptional study was partitioned into three levels: data sets, groups, and samples. A study can include one or many data sets depending on its experimental design [21]. From 56 breast cancer studies (Supplemental Table S1), we have collected 223 breast cancer data sets representing 10,581 breast cancer samples. Primary breast cancer samples within each dataset were grouped by their clinical attributes. Each dataset included at least two groups of tumor samples with various clinical phenotypes (FIG. 1 top panel). For example, the phenotypes related to cancer relapse or poor prognosis include tumor size, nodal involvement, grade, lymphovascular invasion, p53 status, BRCA1 mutation, BRCA2 mutation, estrogen receptor (ER), and human epidermal growth factor receptor 2 (HER2) status [41,42]. Two or more groups per dataset were needed to generate statistical comparisons. A total of 633 significant gene lists (“simple signatures”) from all possible pairwise group comparisons were generated accordingly using a Student's t-test [21]. All 633 “simple signatures” were then stored in a human cancer signature database (HuCaSigDB) that is accessible online (http://seq.mc.vanderbilt.edu/exalt/) [22]. The major procedural steps for extraction of signatures are provided in the Supplemental Methods.

A gene expression signature (“simple signature”) as defined by EXALT is a set of significant genes with their corresponding statistical scores and gene expression direction codes (up or down). Some “simple signatures” are biologically related to breast cancer prognosis, but they were derived from individual transcription profiling studies and are all too often underpowered, truncated, or of low quality. There are inherent limitations for any individual profiling study including small sample size relative to the large number of potential predictors, limitations of technological platforms, sample variation, and bioinformatics or statistical method bias. An underlying assumption we made in formulating this approach is that any individual transcriptional profiling study does not decode an entire expression signature. Rather, these “simple signatures” represent only fragments of a complete and common transcriptional profile (meta-signature).

Identification of a Novel Breast Cancer Meta-Signature

We hypothesized that a meta-signature with improved predictive power could be discovered by data similarity-driven meta-analysis of transcriptional profiles from multiple related studies. EXALT analysis provided the basis for grouping or clustering “simple signatures” sharing significant data similarity. The iterative EXALT process gathered homologous signatures from “simple signatures” and consolidated them into meta-signatures (FIG. 1 middle and lower panel). Briefly, each breast cancer signature was compared with all breast cancer signatures in HuCaSigDB, and signature pairs with significant similarity were grouped together. The intrinsic relationship between pairwise signatures was first determined by gene symbol match and concordance in the direction of gene expression change. Then, a normalized total identity score was calculated based on Q-values from the two signatures. The significant similarity level were determined by simulation analysis [21] as explained in the Supplemental Methods.

We performed iterative EXALT analyses in which all-versus-all signature similarity searches were carried out. More specifically, each of the 633 “simple signatures” from HuCaSigDB served as a seed (also called query or anchored signature) to query all “simple signatures” in HuCaSigDB repeatedly and to bring other homologous signatures together by their common elements (i.e., intrinsic similarities). This iterative process “grouped” or “clustered” signatures based on their similarities (FIG. 1 middle panel). Signature pairs that were sufficiently similar (p<0.05) were linked together to form clusters. After iterative comparisons, each seed signature either remained as a singleton (i.e., a seed signature that self-matched but did not match any other signatures) or formed a cluster with other signatures.

This iterative EXALT process starting with 633 seed signatures resulted in 121 signature clusters and 512 singletons (FIG. 1 middle panel). We focused on eight specific clusters because the eight seed signatures and all other clustered signatures in each of the eight were clearly related to cancer metastasis. The remaining 113 clusters had no consistent and obvious cancer metastasis phenotypes. For the eight metastasis-related clusters, each contained various overlapping signature members associated with phenotypes that are known risk factors for cancer metastasis such as high-grade tumors, ER-negative status, basal-like cell type, and cancer relapse. Of these, we selected the largest signature cluster containing 11 metastasis-related signatures (FIG. 1 and Table 1) [2,3,6,8,11,24,25,34,43,44]. Because each signature in the cluster was derived from a comparison between highly aggressive and less aggressive breast cancers, this comparison yielded a “poor-prognosis” gene signature (Table 1).

Each of the 11 signatures comprises several hundred genes. In order to identify a recurrent and concordant gene expression pattern in the metastatic signature cluster, all genes that comprised the 11 signatures (n=6,526) were assembled into a synthetic signature designated as BRmet. The genes within BRmet were ranked based on recurrent frequency and direction of differential expression (meta-direction) among all 11 signatures. A 100% recurrent frequency was applied to select the top 50 genes for the meta-signature (BRmet50) (FIG. 1 lower panel). Thus, BRmet50 profiles are concordant among all 11 clustered simple signatures (Table 1). BRmet50 genes represent significantly differentially expressed genes not only within their own datasets but also across 11 other related datasets (FIG. 1).

Annotation for BRmet50 genes is provided in Supplemental Table S3. Only five genes in BRmet50 overlapped with BRsig70, and two were found in common with BRsig76. The number of overlapping genes between BRmet50 and the six other cancer signatures (Oncotype DX, TAMR13, Genius, GGI, PAM50, and PIK3CAGS278) is relatively low (1%-27%), suggesting that BRmet50 is a distinct signature. Because BRmet50 was deduced from a cluster of signatures comparing highly aggressive and less aggressive breast cancers, we predicted that BRmet50 would be associated with poor prognoses in breast cancer such as cancer relapse, metastasis, and death. The general prognosis feature of BRmet50 might be different than those of BRsig70/76 (BRmet70 and BRmet76) because they were designed specifically to predict distant metastasis in early-stage breast cancer patients with lymph node-negative status [2,3]. Thus, we realized that neither BRsig70 nor BRsig76 was fully comparable to BRmet50. Rather, they served as prognostic control signatures in this study.

Meta-Validation of BRmet50 in Breast Cancer

Since the BRmet50 was deduced from a signature cluster comparing more and less aggressive cancers, we retrospectively examined the ability of BRmet50 to predict prognosis in 21 datasets, including 11 independent validation datasets not used in the signature clustering process (Table 2).

To examine the stability of the iterative EXALT method and to avoid over-fitting of the nine training datasets, we used a ‘leave-one-out’ cross-validation strategy to deduce nine BRmet50 control signatures for the corresponding nine training datasets. In each leave-one-out trial, the included signatures remained clustered. Furthermore, all BRmet50 control signatures from the ‘leave-one-out’ procedure shared the core set of the 50 genes. We then tested these control meta-signatures in corresponding training datasets (Supplemental Table S2) and found that their prognostic performances were as good as BRmet50 (Table 2). Data suggest that iterative EXALT-based clustering process is a stable and reliable method that is not affected by any particular signature member in the BRmet cluster.

The 11 independent validation datasets were used to evaluate BRmet50 prognosis performance. Log-rank tests were conducted to assess the differences in survival analysis. The p-values from log-rank tests comparing BRmet50, BRsig70, BRsig76, and the six other published cancer signatures (Oncotype DX, TAMR13, Genius, GGI, PAM50 and PIK3CAGS278) are summarized (Table 2 and Supplemental Table S7). Each signature was evaluated for its ability to classify subjects with breast cancer into ‘good’ and ‘poor’ prognostic groups. Expression values for each signature were retrieved from each corresponding dataset, then unsupervised hierarchical clustering was performed using the Spearman rank correlation, and group assignments were determined in each dataset based on the first bifurcation of the clustering dendrograms [40]. BRmet50 distinguished between the good and poor prognostic groups successfully in all datasets (Table 2), while BRsig70 and BRsig76 could not discriminate prognosis groups in four and six datasets respectively. The failure of BRsig70 and BRsig76 to stratify prognostic groups in those datasets persisted after we re-classified samples using the original algorithms (e.g., either the Pearson correlation method [3] or the relapse score method based on weighted Cox's regression coefficient values [2]). Thus, these results were independent of statistical methods. Similar results were also obtained among the six other well-established cancer signatures because none of them could discriminate prognosis groups in all 11 test datasets (Supplemental Table S7). As another performance measure, we calculated the c-index for the cancer signatures in 11 validation datasets (Supplemental Table S7), which is a generalization of the area under the receiver operating characteristic (ROC) curve [45]. The prognostic value (c-index) for BRmet50 and the other cancer signatures were compared. For any given test dataset, BRmet50 c-index is similar to those from the other cancer signatures, suggesting that the BRmet50 and other cancer signatures provide comparable prognostic information.

Performance Measurements in BR1042

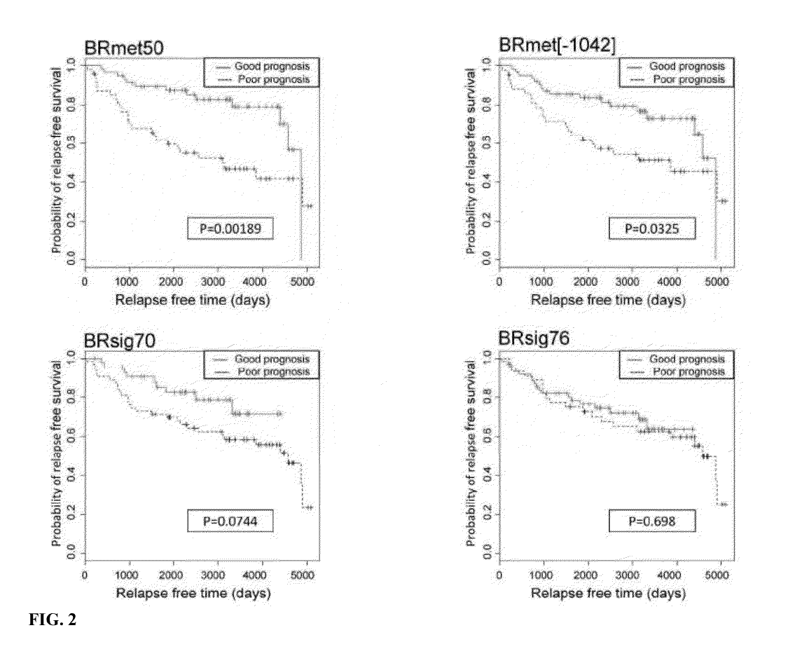

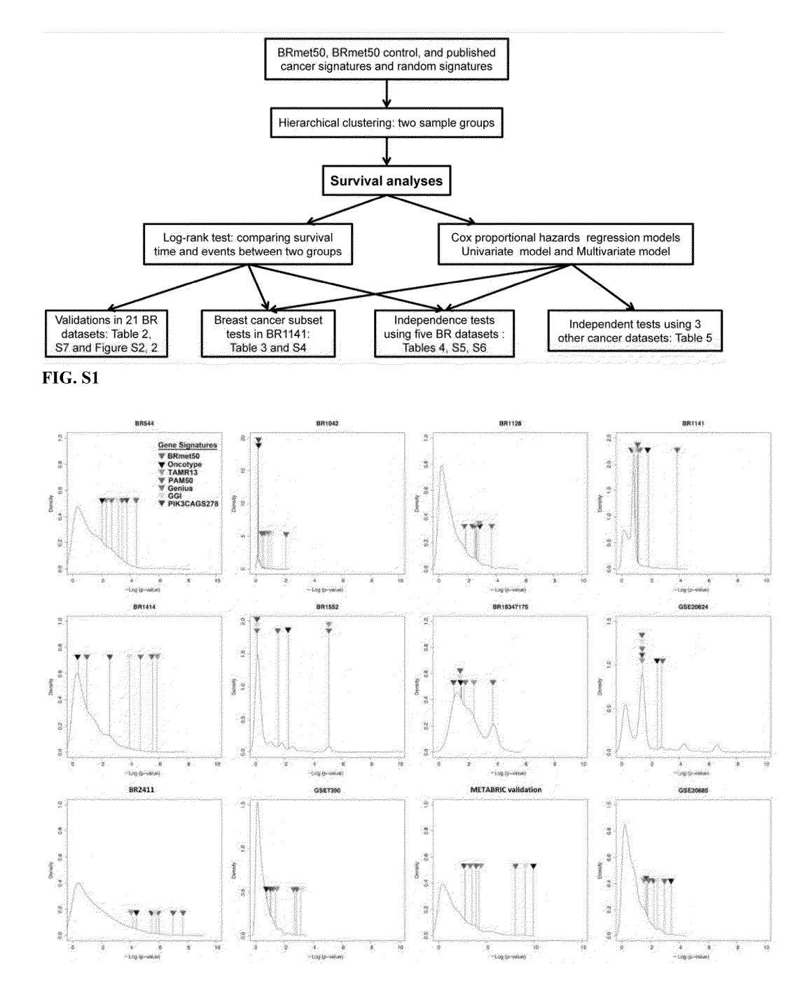

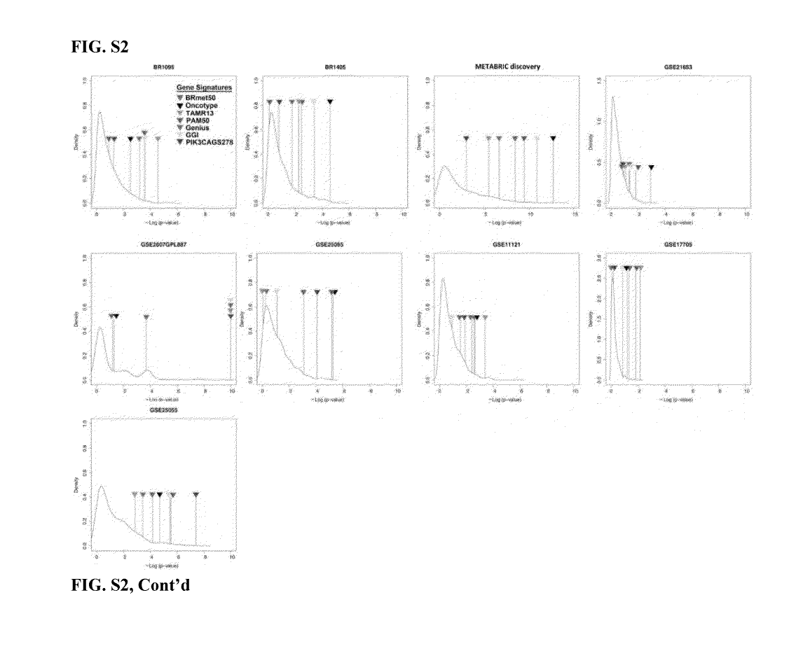

Kaplan-Meier analysis was used to illustrate different relapse-free survival between the previously identified signatures (BRsig70 and BRsig76) and the meta-signature BRmet50 (FIG. 2). The results demonstrate a significant difference in relapse-free survival between the good and poor prognosis groups as predicted for the dataset BR1042 by BRmet50 as well as BRmet50 control signature (BRmet[-1042]) from the leave-one-out process (p<0.05). Among patients for whom BRmet50 predicted a good prognosis, the 10-year rate of relapse-free survival was 79% versus only 47% among those with a poor prognosis (FIG. 2, upper left panel). The risk of relapse predicted by BRmet50 was significantly higher among patients in the poor prognosis group than that among those in the good prognosis group. However, for the same dataset, neither BRsig70 nor BRsig76 distinguished a significant difference in metastasis-free survival between the good and poor prognostic subgroups.

The performance of BRmet50 (c-index: 0.6573, p-value: 0.002) was better than those of BRsig70 and BRsig76 (c-index: 0.5839 or 0.5172, respectively, p-value>0.14) when examining the BR1042 dataset. Our results indicate that the predictive power of BRmet50 is robust and applicable across a wide range of independent datasets.

To assess whether BRmet50 association with prognosis outcome was specific, we generated 1,000 signatures of identical size (50 genes) using randomly selected genes from the human genome. All random signatures were tested in the same panel of 21 test datasets. After 1,000 random permutations of the gene signatures, the p-value distribution (-log p-value) from each test dataset was generated (Figure S2), and p-values from BRmet50 and the six other published cancer signatures were also plotted on the X-axis of the distribution plots (Figure S2).

Although some random signatures are significantly (p<0.05) associated with breast cancer outcomes in various datasets, the associations are stronger for the seven breast cancer signatures in more than half of the test datasets. These control results provide valid statistical support for their prognosis relevance. Furthermore, we noticed that most p-values from BRmet50 were on the far right side of the random p-value distributions (Figure S2). We then compared the patient outcome association of BRmet50 to those of 1,000 random signatures of identical size (Figure S2), and we confirmed that BRmet50 showed a stronger association than the vast majority of (>95%) random signatures. Thus, the probability of obtaining the same p-values as BRmet50 by chance in the same test datasets in Table 2 is significantly low (p<0.05).

Predictive Power of BRmet50 is Independent of Common Clinical and Pathological Covariates

Because dataset BR1141 [6] includes 269 patients with breast cancer and a full panel of common clinical and pathological covariates, we tested whether the association of BRmet50 with poor prognosis outcome was independent of established clinical and pathological criteria using the robust BR1141 dataset examined by Cox proportional-hazards models (Table 3 and Supplemental Table S4). The association between BRmet50 and the risk of poor clinical outcome was significant regardless of tumor size, lymph-node status, or tamoxifen treatment (p<0.05). Furthermore, the BRmet50 could segregate tumors with intermediate differentiation or ER-positive into good and poor prognostic subcategories (hazard ratio for a poor prognosis: 2.5; p≦0.001) but not for those that were ER-negative. Neither BRsig70 nor BRsig76 was capable of stratifying tumors with either good or poor differentiation in any subset of BR1141 except tamoxifen treatment subset (Table 3). Because BR1141 was among the training datasets, we also tested a ‘leave-one-out’ BRmet50 control signature, and found identical significant associations (Supplemental Table S4). The association between BRmet50 and relapse outcome in the BR1141 subset of patients without tamoxifen treatment is further described in the Supplemental result section.

Five of the 21 datasets used for evaluating BRmet50 performance (BR1042, BR1095, BR1128, BR1141, GSE7390) represented 1,183 tumors and had data on a common set of clinicopathologic characteristics including tumor size, grade, lymph node status, and Nottingham Prognostic Index (NPI) [46,47]. Univariate and multivariate analyses of these five validation sets were performed to further evaluate the performance of BRmet50 compared with other prognostic factors, namely, BRsig70, BRsig76, age, tumor size, grade, lymph node status, and NPI. The unadjusted (Supplemental Table S5) and adjusted (Table 4 and Supplemental Table S6) hazard ratios of these factors and signatures were determined.

Univariate Cox proportional-hazards analysis demonstrated that BRsig70, BRsig76, or any individual common prognostic factor (tumor size, grade, lymph node status, or NPI) could not successfully predict cancer prognoses in all five datasets. However, BRmet50 was uniquely able to significantly differentiate tumor samples into two prognostic groups in all five validation sets. The prognostic value of BRmet50 was greater than each of the established risk factors (Supplemental Table S5). For example, optimal unadjusted hazard ratios (HR) (high risk vs. low risk) in BR1128 were 2.8 (95% CI: 1.5-4.9; p<0.001) (BRmet50 control), 1.9 (95% CI: 1.1-3.3; p=0.01) (BRmet70), 2.0 (95% CI: 1.1-3.5; p=0.02) (BRmet76), and 2.2 (95% CI: 1.6-2.9; p<0.01) (NPI), respectively. The data suggested that the BRmet50 was more efficient at predicting relapse-free survival in BR1042, BR1141, and GSE7390 and disease-free survival in BR1095 and BR1128 than established prognostic factors.

Multivariate Cox proportional-hazards analysis was used to determine if BRmet50, BRsig70, or BRsig76 added independent prognostic information to other standard clinicopathological features. In this multivariate Cox proportional-hazards analysis (Table 4), significant associations (p<0.05) were observed in all five test datasets between BRmet50 and patient relapse-free or disease-free time after adjustment for standard clinical covariates. Thus, BRmet50 contributed new and important prognostic information beyond that provided by established clinical predictors. For the most part, BRsig70 and BRsig76 showed no significant associations in these analyses.

Predictive Power of BRmet50 in Other Cancer Types

Because BRmet50 successfully predicted breast cancer prognosis and because some molecular oncogenic events are conserved among multiple cancer types [48], we hypothesized that BRmet50 may represent a conserved transcriptional profile for poor prognosis in multiple cancer types.

To examine the prognostic specificity of BRmet50, we investigated whether BRmet50 could predict prognosis in other epithelial cancers such as colon, lung, or prostate cancer. Three datasets, one for each cancer type: colon cancer (n=73) [49], lung cancer (n=441) [50], and prostate cancer patients (n=596) (Table 5) [51] were subjected to univariate and multivariate analyses. On the basis of gene expression signatures (BRsig70, BRsig76, or BRmet50), 1,110 patient samples were segregated into two groups (Table 5). All three signatures failed to predict cancer relapse in colon cancer [49] (p>0.05). However, BRmet50 but neither BRsig70 nor BRsig76 successfully predicted disease specific survival in prostate cancer and relapse-free survival in lung cancer (p<0.01), suggesting that transcriptional profiles for poor-prognosis may be more conserved in breast, lung, and prostate cancer. In the lung cancer dataset, the good prognosis groups predicted by BRmet50 had the highest relapse-free survival (>40% and p<0.01) among the 3 signatures. We also determined whether the association between the three signatures and the clinical outcomes in patients with prostate, lung, and colon cancer was independent of established clinical and pathological criteria (Table 5). The results suggest that BRmet50 might serve as a prognostic biomarker for both breast and non-breast cancer and may represent a conserved transcriptional profile among multiple cancer types.

Discussion

Data generated by high-throughput transcriptional studies of cancer has rapidly accumulated and there is increasing interest in translating this information into clinical value. Although single-study analysis can be informative, it is often affected by inherent limitations. These limitations can be overcome by combining related independent studies into a meta-analysis. Our study demonstrated that heterogeneous signatures from individual cancer studies can be systematically organized into a meta-signature (BRmet50) based on their intrinsic data similarities by a novel meta-analysis strategy (iterative EXALT). This meta-analysis approach can increase statistical power, minimize false discovery, reduce batch effects, and improve the generalizability of the findings. The value of the BRmet50 signature was evaluated in terms of predicting prognoses in breast and other cancers.

There are two strategies for meta-analysis of transcriptional datasets: data combination and data integration methods. The data combination method is a comprehensive reanalysis of the primary data by merging data from multiple studies [52-60]. This method is powerful because all of the information in the datasets is used. However, this power comes with some risks such as the necessity to model heterogeneity between datasets. Specifically, use of this approach often requires an ad-hoc normalization of the raw data files[61,62] followed by explicitly modeling the inter-study variability [59,63]. The data integration method compares gene lists from any expression platform filtered according to p-values or rank combination [64,65]. The large capacity is not dependent upon the methods used for the initial data processing [66]; heterogeneous datasets become comparable after simplification of raw gene expression values to gene lists, but it comes with the risk that a significant amount of information might be lost. This method has been successfully implemented in a variety of analysis tools such as Venn diagrams, L2L [67], LOLA [68], GeneSigDB [69], Oncomine [70], Connectivity Map (CMAP) [71], and our own novel method called EXALT [21].

Iterative EXALT helped us understand the relationship between the intrinsic signature data similarities and signature-associated phenotypes. When the clustered signature phenotypes in Table 1 were cross-checked with all source phenotypes in Supplemental Table S1, it was confirmed that the datasets with the same sample phenotypes were not necessary to generate signatures with significant data similarity. All data integration methods except EXALT have a shared challenge in how to collect suitable profiling datasets from heterogeneous gene expression studies. These methods typically analyze a limited number of data sets brought together through a prior knowledge-based search (inclusion/exclusion criteria) rather than by intrinsic data similarities [20]. Even though such approaches can ensure that the patient populations or sample phenotypes are similar or homogeneous, they are inadequate given that (1) they can miss valuable datasets and (2) they can include incorrect data sets having no data similarity, resulting in abnormal heterogeneous expression profiles. This characteristic can negatively affect the profile performance, robustness, and applicability. To collect homogeneous datasets for any meta-analysis, it is still a big hurdle when lacking a data-driven quantitative evaluation for inclusion/exclusion criteria [19]. To solve this problem and exploit the enormous wealth of available data to their full potential, iterative EXALT can systematically integrate available transcriptional datasets in public domains (Supplemental Table S1) based on intrinsic data similarities. The unique processes performed by the iterative EXALT method include gathering homogeneous signatures for meta-analysis (Table 1), consolidating homogeneous signatures, and discovering reliable and recurrent meta-signatures for given diseases or biologically related phenotypes (FIG. 1). These important features are not present in our previous EXALT program [21,22], nor can they be found in any other data integration methods.

The power of the iterative EXALT is illustrated in the identification of a meta-signature (BRmet50). In our meta-validation of BRmet50, we found that distinct gene expression signatures have a common significant predictive value in more than half of the breast cancer studies (Table 2, Supplemental Table S7, and Figure S2). This agreement supports the notion that the limited overlap in gene identity among gene expression profiles does not affect similar prognostic performance in breast cancer [72]. However, unlike other studies in which only a few test datasets were examined [73,74], our current study included a large number of test datasets. We found that some well-established cancer signatures were not significant predictors in several published breast cancer survival studies (Table 2 and Supplemental Table S7). Further, when adjusted for major prognostic clinical covariates, neither BRsig70 nor BRsig76 was able to discriminate between good and poor prognosis groups in multiple breast cancer datasets (Table 4). This observation agrees with the notion that BRsig70 is a predictor of early relapse and is of limited clinical utility in breast cancers [11,15,75-80]. One explanation is that BRsig70 and BRsig76 had been previously validated only in a few datasets with the selected patient subsets (e.g., patients with lymph-node-negative status) [11,75,81]. A large prospective clinical trial (MINDACT) is now being carried out [82] to test whether BRsig70 can predict prognosis in patients with node-negative as well as those patients with one to three positive lymph node to avoid chemotherapy [15]. Our results emphasize the need to perform additional validation studies of transcriptional biomarkers, including a demonstration of their value beyond common histopathological predictors [5,83], for extrapolation to a more general patient population [84-88]. A meta-analysis strategy combining both discovery and validation of transcriptional biomarkers may be well-suited to accomplish these goals.

A previous report [73] suggested that a large percentage (>50%) of random gene expression signatures were significantly associated with breast cancer outcome in two breast cancer datasets (designated here as BR2411[11] and BR1141[6]). We generated 1,000 random signatures with identical in size to BRmet50 from the human genome and examined them using 21 validation datasets (Supplemental Figure S2). Based on the random p-value distributions, we found that the distributions were heterogeneous. Some datasets such as BR18347175 and GSE20624 had unusual skewed distributions of random p-values and a high percentage (50% or higher) of random signatures that were significantly associated with breast cancer outcome at p<0.05. However, for the majority of the other validation datasets, outcome association of BRmet50 and most published cancer signatures showed stronger associations than the median of random signatures. On average, the association of BRmet50 with disease outcome was stronger than that of the top 5% random signatures (Supplemental Figure S2).

One important observation from this large scale validation of results is that a random signature may produce significant outcome associations (p<0.05) in a small number of test datasets, but it is still very difficult for a random signature to repeatedly yield significant results by chance in a majority of 21 test datasets. Out of 1,000 random signatures, there were only 13 (1.3% of random signatures) that generated significant predictions (p<0.05) from more than 10 out of 21-test datasets (>50%). However, for the same 21 validation datasets, 100% of the tests of BRmet50 and more than 50% of the tests of the six other known cancer signatures were significant. Abiding to this criterion, the probability that a random signature achieves the similar level of performance as the BRmet50 by chance is low (p<0.013). Clearly, our study emphasizes the importance of large-scale validation tests.

A 21-gene signature (Oncotype DX) is a diagnostic test that quantifies the likelihood of relapse of tamoxifen-treated, lymph node-negative breast cancer using a recurrence score method [4]. The recurrence score is derived from the RT-PCR based reference-normalized expression measurements for 16 cancer-related genes. The panel of 21 genes in Oncotype DX includes some well-known biomarker genes for breast cancer subtypes and prognosis prediction such as Ki67, HER2, ER, and PGR. This has raised concern about whether it truly adds independent prognostic information beyond other standard clinicopathological covariates [5,83]. We did not apply the Oncotype recurrence score formula directly to gene expression values described in this study. In order to make comparisons between BRmet50 and this widely used prognostic marker, we examined the prognosis prediction values of Oncotype DX signature and the other six well-known cancer signatures in all 21 test datasets (Supplemental Figure S2 and Supplemental Table S7) using random signature simulations as negative controls. The results suggest that Oncotype DX is a strong predictor with significant predictions in 80% of test datasets.

Because breast cancer is such a heterogeneous disease, most recent studies have taken the molecular heterogeneity of breast cancer into account in their predictions [7,89]. As a general prognosis predictor in cancer, BRmet50 is a meta-signature derived from datasets representing heterogeneous cancer subtypes, and BRmet50 therefore represents mixed gene expression profiles of various breast cancer subtypes. During the univariate analysis of breast cancer subtypes (Table 3), we noticed that BRmet50 could be used to segregate ER-positive (luminal tumors) and intermediate grade tumors regardless of tumor size and lymph-node status into good and poor prognostic subcategories (hazard ratio for a poor prognosis: 2.5; p<0.001) but not for those with ER-negative status or those with high-grade (Table 3). These prognostic features are consistent with those of many other breast cancer prognostic signatures [74,90-94] but different than those of subtype-specific prognostic predictors (GENIUS) that can be applied to breast cancer samples with ER-negative or HER2-negative status[7]. When BRmet50 was used in the subtype classification model [89] we found that BRmet50 could inform subtype classification but its prediction strength was not as robust as the three-gene model[89].

In summary, we have developed and demonstrated the utility of a novel data similarity-based meta-analysis strategy for deducing a transcriptional meta-signature with enhanced prognostic value in breast cancer. We report a novel meta-signature, BRmet50, which has a superior capability to predict clinical outcomes in 21 breast cancer transcriptional profiling datasets. Furthermore, BRmet50 can distinguish prognostic subsets of patients with ER-positive breast cancer or intermediate-grade breast cancer regardless of lymph-node and tamoxifen treatment status. Finally, we demonstrated that BRmet50 has predictive value in other cancer types (prostate and lung), suggesting that different cancers may share common transcriptional elements that influence their clinical behaviors. Additional prospective studies will be valuable in determining the clinical value of BRmet50 in breast cancer patient subsets and other cancers.

Abbreviations

BRmet50: 50-gene signature; BRsig70: the 70-gene signature in breast cancer or MammaPrint; BRsig76: the 76-gene signature in breast cancer; CI: confidence interval; ER: estrogen receptor); EXALT: EXpression AnaLysis Tool; GEO: Gene Gxpression Omnibus; HR: hazard ratio; NCBI: the National Center for Biotechnology Information; NPI: Nottingham Prognostic Index; Oncotype DX: the 21-gene signature; ROC: receiver operating characteristic.

Supplemental Methods and Data

EXALT analysis. We previously described EXALT as a novel analytical system that can compare gene-expression signatures across studies without limitations imposed by different technological platforms, different laboratories, or different species [1]. Rather than compare raw data, EXALT implements a search paradigm that matches gene-expression signatures deduced from preprocessed (i.e., normalized and background subtracted) data like those deposited in the Gene-expression Omnibus (GEO) database. Because of this feature, EXALT can compare data generated by any platform and is independent of the methods used in the initial data processing. The output from EXALT provides similarity scores and statistical confidence levels for each signature match allowing a rapid perusal of relationships between the query data and the entries in a database of signatures from other experiments. One database (HuCaSigDB) that was used extensively in this study holds published transcriptional profiling studies of human cancer (see below).

Signature Extraction and Database.

We processed 223 published breast cancer gene-expression profiling datasets from 56 published breast cancer studies to enable extraction of expression signatures (Supplemental Table S1). We used a four-step process to extract gene-expression signatures from individual data sets, which we described previously [1]. First, data were formatted into a common data type. Second, we tested each gene for significant differential expression by comparing two groups of samples and calculating a Student's t-statistic. The significant gene p-value (false positive rate) determined for each significant gene was adjusted by the false discovery rate (FDR) method using q-values [2]. Third, a list of significant genes with Q-values □0.2 was generated, and then the reciprocal logarithms of the Q-values (-log [Q-value]) were calculated to get the Q-scores. Finally, gene-expression signatures were generated in the form of a list of “triplets”, each defined as a gene symbol-direction code-Q-score. The direction code was determined by the relative difference in expression between two group means and could have one of three values (U, up; D, down; X, uncertain). Signatures were stored in a relational database (HuCaSigDB) linked with clinical outcome data and experimental features from the original studies. Thus, a gene expression signature as defined by EXALT is a set of significant genes with their corresponding statistical scores and direction codes. In essence, a signature (or group of signatures) represents a statistically validated ‘fingerprint’ associated with a biological observation made from a gene expression experiment.

Determination of Signature Similarity.

In the iterative EXALT analysis, each query signature was compared with every subject signature in HuCaSigDB using an algorithm we described previously [1]. For each pair of query and subject signatures with lengths Lq and Ls, a total identity score (TIS) was computed in three steps. First, the signatures were aligned by matching gene symbols. Then the direction codes (U, D, or X) for matching genes were determined to be concordant (i.e., U-U, or D-D), discordant (i.e., U-D), or uncertain (i.e. presence of direction code X in either query or subject). Next, the Q scores were summed separately for concordant and discordant matches to give a positive identity score (PIS) and a negative identity score (NIS), respectively, by the formulas:

PIS=Σ(Siq+Sis), i=1, . . . N

NIS=Σ(Sjg+Sjs), j=1, . . . M

where N and M are numbers of concordant and discordant matches, respectively, and Siq and Sis (Sjq and Sjs) are Q-scores for the ith concordant (jth discordant) match in the query and subject signatures. The NIS score was assigned a negative value because of its opposite direction from PIS scores. Matches with at least one direction code of X and all non-matching genes were excluded from the identity score calculations. Finally, the total identity score (TIS) was computed as the absolute value of the sum of the PIS and NIS divided by the sum of signature lengths (Lq+Ls) using: TIS=|PIS+NIS|/(Lq+Ls).

Defining Significance Level.

We performed simulations to determine the statistical significance of the TIS values. We generated 1000 random query signatures and computed the TIS between each query signature and each subject signature in HuCaSigDB. The random query signatures had similar properties (length distribution, gene symbol frequency, uniqueness) to those of the actual data. The results suggested that the TIS score correlated well with query signature length. To adjust for the influence of query signature length, we derived the mean and standard deviation (SD) of the TIS as functions of query length and then normalized the TIS by converting it to its Z-score using the formula: ZTIS-score=(TIS−mean)/SD, where mean and SD are functions of query length. This enabled us to generate an empirical distribution of ZTIS-scores. For a real query, we followed the same procedure to calculate the ZTIS-score, and compared it with the empirical distribution to estimate its corresponding query p-value. A query is statistically significant if its p-value is ≦0.01.

Identification of Meta-Signature BRmet50.

From the initial signature clusters, a single cluster was identified for its large group of signatures with common clinical phenotypes related to cancer prognosis. This cluster consisted of 11 metastasis-related signatures (Table 1). Ten out of 11 signatures shared significant similarity with the same query signature (Sig2411 in Table 1) derived from a comparison between the non-metastasis and metastasis sample groups in breast cancer [3]. Furthermore, all 11 signatures in the cluster were related to known metastasis risk factors including high-grade tumors, ER-negative status, basal-like cell type, and cancer relapse (Table 1).

To identify a recurrent and concordant gene-expression pattern in the metastatic signature cluster, we assembled the 11 signatures representing 2,034 tumor samples into a single meta-signature designated as BRmet. The genes in BRmet were ranked based on their recurrent frequencies and differential expression directions among all 11 signatures. Fifty genes (BRmet50) were present in all 11 signatures and had concordant expression vectors (annotation for these 50 genes is provided in Supplemental Table S3). Only five genes in BRmet50 overlapped with BRsig70 and two were found to be in common with BRsig76, suggesting that BRmet50 was a distinct signature. Among the 50 genes, 39 were up-regulated while the other 11 genes exhibited lower expression in aggressive tumors. Because BRmet50 was deduced from a cluster of signatures comparing highly aggressive breast cancer with less aggressive one, we predicted that BRmet50 would be associated with a poor prognosis.

Cross Validation Studies Using a Leave-One-Out Approach.

We retrospectively examined BRsig70, BRsig76, and BRmet50 using 21 datasets as presented in Table 2 for the prediction of disease prognosis in breast cancer subjects. Among the 21 validation datasets, 10 of them were the aforementioned datasets used for discovering BRmet50 in Table 1 and FIG. 1. To examine the stability of the iterative EXALT system and to avoid over-fitting the nine datasets, we derived nine control signatures for BRmet50 and their nine corresponding test datasets (Table 2 and Supplemental Table S2) using a leave-one-out cross validation strategy. For instance, one signature member (Sig2411) in the cluster was left out intentionally during the iterative EXALT processes. The remaining 10 signatures comprised a new cluster that was then used to assemble a new meta-signature designated as BRmet[-2411]. The left-out signature ID (Sig2411) was used to label the meta-signature and its original source dataset. For example, BRmet[-2411] indicates that the Sig2411 was dropped out before the clustering and assembling process of BRmet[-2411], and BR2411 is its source dataset. BRmet[-2411] is a special case of BRmet50. Both BRmet50 and BRmet[-2411] share the core set of the top 50 signature genes, but BRmet[-2411] has an additional 12 genes. We repeated this procedure until each signature had been was left out once. Therefore, this repetitive cross-validation procedure produced nine test BRmet signatures (Supplemental Table S2). Nine corresponding breast cancer test datasets were also made. Two datasets (BR907 and BR1224) were dropped because they had either insufficient survival data size or sample size thus failing to meet our inclusion criteria.

The independent survival validation set included 11 GEO datasets (Table 2). During survival analyses with control signatures, we only tested the control signature (e.g., BRmet[-2411]) in its corresponding dataset (e.g., BR2411) (Supplemental Table S2). However, we retrospectively tested the BRsig70, BRsig76, and BRmet50 in all 21 breast cancer test datasets for the prediction of metastasis-free survival.

BRmet50 Performance is Independent of Tamoxifen Treatment.

To investigate whether tamoxifen treatment had any confounding effects on the utility of BRmet50, we divided the BR1141 dataset into two groups of patients based on their exposure to tamoxifen treatment and focused our analysis on the 108 patients who never received tamoxifen [4]. We used this subgroup to evaluate whether BRmet50 was independent of the confounding effects of tamoxifen treatment. Using Spearman correlation clustering and Cox proportional-hazards models, the 108 patients were stratified by BRmet50 into two groups: 47 patients with good prognosis and 61 patients with poor prognosis. The association between BRmet50 and relapse outcome in these 108 patients was significant (hazard ratio=2.6, p<0.005, Table 3). In contrast, neither BRsig70 nor BRsig76 was able to successfully divide the 108 patients into two prognostic groups.

Annotation of BRmet50 Genes.

The BRmet50 gene list includes genes involved in maintaining the cell cycle (23 genes), DNA replication (9 genes), proliferation (7 genes), and cellular motility/assembly (5 genes). These functional processes contribute to tumorigenesis, cancer progression, and metastasis. More than half of the BRmet50 genes relate to cell growth (DNA replication, cell cycle, and proliferation), which is a known component of breast cancer prognostic signatures [5]. The annotated results indicate that the BRmet50 gene meta-directions and functions correlate with their roles in cancer progression and metastasis. For example, up-regulated genes are often involved in tumor progression and have functional roles in cell cycle, DNA replication, or proliferation. Tumor suppressor genes with functional roles in anti-proliferation or cellular movement/assembly are down-regulated in aggressive tumors. On the basis of these observations, we can infer the differential expression of BRmet50 genes in clinical samples and their roles in cancer pathogenesis based on their meta-directions in BRmet50 and their physiologic functions.

Although our approach uses BRmet50 as a biomarker for disease progression, these genes may have more than just prognostic value and may possibly represent common key regulators of metastatic disease. We examined the classification of the 50 genes by their functions and relevance to cancer. The resulting information can be found in Supplemental Table S3. It is not surprising that some of the BRmet50 genes are known to be involved in tumor progression. For example, 30% (15/50) of BRmet50 genes were reported previously as tumor progression genes for multiple cancer types [6-12]. They include KPNA2 and TYMS for breast cancer [13,14]; UBE2C, CCNB1, RRM2, and CCNB2 for lung cancer [15-18]; and NDC80 for prostate cancer [19]. The data support our result in Table 5 indicating that BRmet50 represents a conserved transcription profile across multiple cancer types. The differential expression directions of these 50 genes are nearly 100% concordant (i.e., up- or down-regulated at the same time) across all 11 signatures from 2,034 breast tumor samples (Table 1). A total of 39 out of the 50 genes were up-regulated, and 11 out of 50 genes were expressed at a lower level in aggressive tumors. Some of the BRmet50 gene expression profiles (29/50) have been supported by prior works (Supplemental Table S3), while gene expression directions for the other 20 have not yet been reported. Only one gene, GTPBP4, was found to have the opposite expression direction in the literature [20]. Among the 39 up-regulated genes, 14 genes have been previously reported to contribute to cancer progression and/or metastasis. Two down-regulated BRmet50 genes are known tumor suppressors (BTG2 and SCUBE2) in breast carcinomas [21,22].

Comparison of Meta-Analysis Methods for Gene-Expression Profiles.

EXALT has a unique signature encoding format for summarizing transcriptional results, large signature databases, and a powerful signature search engine [1,23]. The iterative EXALT method has additional novel features. These include gathering homologous signatures for meta-analysis, consolidating heterogeneous signatures, and discovering reliable and recurrent meta-signatures for disease prognosis. These important novel features are not present in the previous EXALT program, nor can they be found in any other meta-analysis methods.

Meta-analysis of gene-expression data for the purposes of developing clinically relevant transcriptional biomarkers has many challenges that have not been previously overcome. To illustrate a new paradigm for discovering transcriptional biomarkers, we have demonstrated that a meta-analysis strategy using iterative EXALT was capable of deducing novel meta-signatures using data from multiple independent transcriptional profiling studies. This was highlighted by the identification of a 50-gene profile (BRmet50) for predicting increased risks of metastasis and poor clinical outcome in breast cancer. We subsequently showed that this signature outperformed BRsig70 and BRsig76, especially when clinical covariates were considered. Specifically, BRmet50 is capable of predicting breast cancer clinical outcome independent of tumor size, lymph-node status, tamoxifen treatment, histological grade, and ER status. Furthermore, BRmet50, not BRsig70 or BRsig76, had predictive value in lung and prostate cancer, suggesting that this meta-signature may be a marker for common and important transcriptional events shared by multiple neoplasms. These findings demonstrate the utility of iterative EXALT meta-analysis for identifying novel transcriptional biomarkers with predictive power in breast cancer and other cancers.

The success of our approach may be mainly attributable to a large sample size and our unique meta-analysis strategy. Like any other gene-expression data analysis, simple signatures are extracted from clinically defined group comparisons, but iterative EXALT has performed integrated analysis to extract simple signatures using a much larger sample size. In this study alone, a total of 223 datasets containing 10,581 tumor samples (discovery set) were included. Next, iterative EXALT carried out signature clustering to identify a signature cluster. The simple signatures in the cluster are biologically related to breast cancer prognosis and share significant data similarity, but they represent individual gene-expression profiles. To identify a recurrent and concordant gene-expression pattern, iterative EXALT assembled these 11 homologous signatures into a synthetic signature designated as the breast cancer metastasis meta-signature (BRmet50). Therefore, BRmet50 is neither a replicate of the original signatures nor simple intersections of the overlapping genes from multiple clustered signatures. Rather, it conceptually represents an expanded expression profile with inclusion of the expression directions and confidence levels of the individual signature genes.

For meta-analysis of transcriptional profiles, other computational platforms, such as L2L [24], LOLA [25], GeneSigDB [26], Oncomine [27], and Connectivity Map (CMAP) [28], have been described but have not been implemented successfully to extract meta-signatures. These methods typically analyze a limited number of data sets brought together through a prior knowledge-based search (inclusion/exclusion criteria) rather than by intrinsic data similarities. Such approaches are inadequate given that (1) they can miss valuable datasets and (2) they can include incorrect data sets, resulting in abnormal heterogeneous expression profiles. This characteristic can negatively affect the profile performance, robustness, and applicability. For example, the Connectivity Map (CMAP) is a gene-expression signature database application derived using Gene Set Enrichment Analysis (GSEA) [28,29]. As a data integration meta-analysis application, GSEA can compare or annotate input signatures using molecular knowledge (gene loci or gene signature-related pathways), but there is not an accompanying expression signature database (e.g., GEO or Human Cancer) in GSEA. CMAP has a signature database that focuses exclusively on drug-treated cell line expression data from one platform using a single source. In Oncomine, meta-analysis is enabled by searching for a gene name or experimental keyword rather than by signature similarity [30]. This approach does not support signature comparisons across datasets or clustering of signatures. Other approaches for meta-analysis of transcriptional profiles have similar limitations.

Throughout this document, various references are mentioned. All such references are incorporated herein by reference, including the references set forth in the following list:

### REFERENCES

1. Sotiriou C and Pusztai L. (2009) Gene-expression signatures in breast cancer. N Engl J Med 360: 790-800.
2. Wang Y, Klijn J G, Zhang Y, Sieuwerts A M, Look M P, et al. (2005) Gene-expression profiles to predict distant metastasis of lymph-node-negative primary breast cancer. Lancet 365: 671-679.
3. van', V, Dai H, van d, V, He Y D, Hart A A, et al. (2002) Gene expression profiling predicts clinical outcome of breast cancer. Nature 415: 530-536.
4. Paik S, Shak S, Tang G, Kim C, Baker J, et al. (2004) A multigene assay to predict recurrence of tamoxifen-treated, node-negative breast cancer. N Engl J Med 351: 2817-2826.
5. Flanagan M B, Dabbs D J, Brufsky A M, Beriwal S, and Bhargava R. (2008) Histopathologic variables predict Oncotype DX recurrence score. Mod Pathol 21: 1255-1261.
6. Loi S, Haibe-Kains B, Desmedt C, Wirapati P, Lallemand F, et al. (2008) Predicting prognosis using molecular profiling in estrogen receptor-positive breast cancer treated with tamoxifen. BMC Genomics 9: 239.
7. Haibe-Kains B, Desmedt C, Rothe F, Piccart M, Sotiriou C, et al. (2010) A fuzzy gene expression-based computational approach improves breast cancer prognostication. Genome Biol 11: R18.
8. Sotiriou C, Wirapati P, Loi S, Harris A, Fox S, et al. (2006) Gene expression profiling in breast cancer: understanding the molecular basis of histologic grade to improve prognosis. J Natl Cancer Inst 98: 262-272.
9. Parker J S, Mullins M, Cheang M C, Leung S, Voduc D, et al. (2009) Supervised risk predictor of breast cancer based on intrinsic subtypes. J Clin Oncol 27: 1160-1167.
10. Loi S, Haibe-Kains B, Majjaj S, Lallemand F, Durbecq V, et al. (2010) PIK3CA mutations associated with gene signature of low mTORC1 signaling and better outcomes in estrogen receptor-positive breast cancer. Proc Natl Acad Sci USA 107: 10208-10213.
11. van d, V, He Y D, van't Veer L J, Dai H, Hart A A, et al. (2002) A gene-expression signature as a predictor of survival in breast cancer. N Engl J Med 347: 1999-2009.
12. Ioannidis J P, Allison D B, Ball C A, Coulibaly I, Cui X, et al. (2009) Repeatability of published microarray gene expression analyses. Nat Genet 41: 149-155.
13. Ioannidis J P. (2005) Microarrays and molecular research: noise discovery? Lancet 365: 454-455.
14. Ransohoff D F. (2004) Rules of evidence for cancer molecular-marker discovery and validation. Nat Rev Cancer 4: 309-314.
15. Weigelt B, Baehner F L, and Reis-Filho J S. (2010) The contribution of gene expression profiling to breast cancer classification, prognostication and prediction: a retrospective of the last decade. J Pathol 220: 263-280.
16. Reis-Filho J S, Westbury C, and Pierga J Y. (2006) The impact of expression profiling on prognostic and predictive testing in breast cancer. J Clin Pathol 59: 225-231.
17. Sontrop H M, Verhaegh W F, Reinders M J, and Moerland P D. (2011) An evaluation protocol for subtype-specific breast cancer event prediction. PLoS One 6: e21681.
18. Teschendorff A E, Naderi A, Barbosa-Morais N L, Pinder S E, Ellis I O, et al. (2006) A consensus prognostic gene expression classifier for ER positive breast cancer. Genome Biol 7: R101.
19. Tseng G C, Ghosh D, and Feingold E. (2012) Comprehensive literature review and statistical considerations for microarray meta-analysis. Nucleic Acids Res.
20. Ramasamy A, Mondry A, Holmes C C, and Altman D G. (2008) Key issues in conducting a meta-analysis of gene expression microarray datasets. PLoS Med 5: e184.
21. Yi Y, Li C, Miller C, and George A L, Jr. (2007) Strategy for encoding and comparison of gene expression signatures. Genome Biol 8: R133.
22. Wu J, Qiu Q, Xie L, Fullerton J, Yu J, et al. (2009) Web-based interrogation of gene expression signatures using EXALT. BMC Bioinformatics 10: 420.
23. Barrett T, Suzek T O, Troup D B, Wilhite S E, Ngau W C, et al. (2005) NCBI GEO: mining millions of expression profiles—database and tools. Nucleic Acids Res 33: D562-D566.
24. Miller L D, Smeds J, George J, Vega V B, Vergara L, et al. (2005) An expression signature for p53 status in human breast cancer predicts mutation status, transcriptional effects, and patient survival. Proc Natl Acad Sci USA 102: 13550-13555.
25. Ivshina A V, George J, Senko O, Mow B, Putti T C, et al. (2006) Genetic reclassification of histologic grade delineates new clinical subtypes of breast cancer. Cancer Res 66: 10292-10301.
26. Hatzis C, Pusztai L, Valero V, Booser D J, Esserman L, et al. (2011) A genomic predictor of response and survival following taxane-anthracycline chemotherapy for invasive breast cancer. JAMA 305: 1873-1881.
27. Kao K J, Chang K M, Hsu H C, and Huang A T. (2011) Correlation of microarray-based breast cancer molecular subtypes and clinical outcomes: implications for treatment optimization. BMC Cancer 11: 143.
28. Anders C K, Fan C, Parker J S, Carey L A, Blackwell K L, et al. (2011) Breast carcinomas arising at a young age: unique biology or a surrogate for aggressive intrinsic subtypes? J Clin Oncol 29: e18-e20.
29. Symmans W F, Hatzis C, Sotiriou C, Andre F, Peintinger F, et al. (2010) Genomic index of sensitivity to endocrine therapy for breast cancer. J Clin Oncol 28: 4111-4119.
30. Sabatier R, Finetti P, Cervera N, Lambaudie E, Esterni B, et al. (2011) A gene expression signature identifies two prognostic subgroups of basal breast cancer. Breast Cancer Res Treat 126: 407-420.
31. Schmidt M, Bohm D, von T C, Steiner E, Puhl A, et al. (2008) The humoral immune system has a key prognostic impact in node-negative breast cancer. Cancer Res 68: 5405-5413.
32. Desmedt C, Piette F, Loi S, Wang Y, Lallemand F, et al. (2007) Strong time dependence of the 76-gene prognostic signature for node-negative breast cancer patients in the TRANSBIG multicenter independent validation series. Clin Cancer Res 13: 3207-3214.
33. Perreard L, Fan C, Quackenbush J F, Mullins M, Gauthier N P, et al. (2006) Classification and risk stratification of invasive breast carcinomas using a real-time quantitative RT-PCR assay. Breast Cancer Res 8: R23.
34. Hu Z, Fan C, Oh D S, Marron J S, He X, et al. (2006) The molecular portraits of breast tumors are conserved across microarray platforms. BMC Genomics 7: 96.
35. Chanrion M, Negre V, Fontaine H, Salvetat N, Bibeau F, et al. (2008) A gene expression signature that can predict the recurrence of tamoxifen-treated primary breast cancer. Clin Cancer Res 14: 1744-1752.
36. Curtis C, Shah S P, Chin S F, Turashvili G, Rueda O M, et al. (2012) The genomic and transcriptomic architecture of 2,000 breast tumours reveals novel subgroups. Nature 486: 346-352.
37. Hsieh F Y and Lavori P W. (2000) Sample-size calculations for the Cox proportional hazards regression model with nonbinary covariates. Control Clin Trials 21: 552-560.
38. Shih J H. (1995) Sample size calculation for complex clinical trials with survival endpoints. Control Clin Trials 16: 395-407.
39. Cantor A B. (1992) Sample size calculations for the log rank test: a Gompertz model approach. J Clin Epidemiol 45: 1131-1136.
40. Lukes L, Crawford N P, Walker R, and Hunter K W. (2009) The origins of breast cancer prognostic gene expression profiles. Cancer Res 69: 310-318.
41. Kennecke H, Yerushalmi R, Woods R, Cheang M C, Voduc D, et al. (2010) Metastatic behavior of breast cancer subtypes. J Clin Oncol 28: 3271-3277.
42. Haffty B G, Yang Q, Reiss M, Kearney T, Higgins S A, et al. (2006) Locoregional relapse and distant metastasis in conservatively managed triple negative early-stage breast cancer. J Clin Oncol 24: 5652-5657.
43. Oh D S, Troester M A, Usary J, Hu Z, He X, et al. (2006) Estrogen-regulated genes predict survival in hormone receptor-positive breast cancers. J Clin Oncol 24: 1656-1664.
44. Herschkowitz J I, He X, Fan C, and Perou C M. (2008) The functional loss of the retinoblastoma tumour suppressor is a common event in basal-like and luminal B breast carcinomas. Breast Cancer Res 10: R75.
45. Clark T G, Bradburn M J, Love S B, and Altman D G. (2003) Survival analysis part IV: further concepts and methods in survival analysis. Br J Cancer 89: 781-786.
46. Nedumpara T, Jonker L, and Williams M R. (2011) Impact of immediate breast reconstruction on breast cancer recurrence and survival. Breast.
47. Rakha E A, Reis-Filho J S, Baehner F, Dabbs D J, Decker T, et al. (2010) Breast cancer prognostic classification in the molecular era: the role of histological grade. Breast Cancer Res 12: 207.
48. Albihn A, Johnsen J I, and Henriksson M A. (2010) MYC in oncogenesis and as a target for cancer therapies. Adv Cancer Res 107: 163-224.
49. Garman K S, Acharya C R, Edelman E, Grade M, Gaedcke J, et al. (2008) A genomic approach to colon cancer risk stratification yields biologic insights into therapeutic opportunities. Proc Natl Acad Sci USA 105: 19432-19437.
50. Shedden K, Taylor J M, Enkemann S A, Tsao M S, Yeatman T J, et al. (2008) Gene expression-based survival prediction in lung adenocarcinoma: a multi-site, blinded validation study. Nat Med 14: 822-827.
51. Nakagawa T, Kollmeyer T M, Morlan B W, Anderson S K, Bergstralh E J, et al. (2008) A tissue biomarker panel predicting systemic progression after PSA recurrence post-definitive prostate cancer therapy. PLoS One 3: e2318.
52. Szabo P M, Tamasi V, Molnar V, Andrasfalvy M, Tombol Z, et al. (2010) Meta-analysis of adrenocortical tumour genomics data: novel pathogenic pathways revealed. Oncogene 29: 3163-3172.
53. Stevens J R and Nicholas G. (2009) metahdep: meta-analysis of hierarchically dependent gene expression studies. Bioinformatics 25: 2619-2620.
54. Bisognin A, Coppe A, Ferrari F, Risso D, Romualdi C, et al. (2009) A-MADMAN: annotation-based microarray data meta-analysis tool. BMC Bioinformatics 10: 201.
55. Wren J D. (2009) A global meta-analysis of microarray expression data to predict unknown gene functions and estimate the literature-data divide. Bioinformatics 25: 1694-1701.
56. Alles M C, Gardiner-Garden M, Nott D J, Wang Y, Foekens J A, et al. (2009) Meta-analysis and gene set enrichment relative to er status reveal elevated activity of MYC and E2F in the “basal” breast cancer subgroup. PLoS One 4: e4710.
57. Ma S and Huang J. (2009) Regularized gene selection in cancer microarray meta-analysis. BMC Bioinformatics 10: 1.
58. Ochsner S A, Steffen D L, Hilsenbeck S G, Chen E S, Watkins C, et al. (2009) GEMS (Gene Expression MetaSignatures), a Web resource for querying meta-analysis of expression microarray datasets: 17beta-estradiol in MCF-7 cells. Cancer Res 69: 23-26.
59. Borozan I, Chen L, Paeper B, Heathcote J E, Edwards A M, et al. (2008) MAID: an effect size based model for microarray data integration across laboratories and platforms. BMC Bioinformatics 9: 305.
60. Smith D D, Saetrom P, Snove 0, Jr., Lundberg C, Rivas G E, et al. (2008) Meta-analysis of breast cancer microarray studies in conjunction with conserved cis-elements suggest patterns for coordinate regulation. BMC Bioinformatics 9: 63.
61. Bisognin A, Coppe A, Ferrari F, Risso D, Romualdi C, et al. (2009) A-MADMAN: annotation-based microarray data meta-analysis tool. BMC Bioinformatics 10: 201.
62. Cahan P, Rovegno F, Mooney D, Newman J C, St L G, III, et al. (2007) Meta-analysis of microarray results: challenges, opportunities, and recommendations for standardization. Gene 401: 12-18.
63. Choi J K, Yu U, Kim S, and Yoo O J. (2003) Combining multiple microarray studies and modeling interstudy variation. Bioinformatics 19 Suppl 1: i84-i90.
64. Rhodes D R, Barrette T R, Rubin M A, Ghosh D, and Chinnaiyan A M. (2002) Meta-analysis of microarrays: interstudy validation of gene expression profiles reveals pathway dysregulation in prostate cancer. Cancer Res 62: 4427-4433.
65. DeConde R P, Hawley S, Falcon S, Clegg N, Knudsen B, et al. (2006) Combining results of microarray experiments: a rank aggregation approach. Stat Appl Genet Mol Biol 5: Article 15.
66. Rhodes D R, Yu J, Shanker K, Deshpande N, Varambally R, et al. (2004) Large-scale meta-analysis of cancer microarray data identifies common transcriptional profiles of neoplastic transformation and progression. Proc Natl Acad Sci USA 101: 9309-9314.
67. Newman J C and Weiner A M. (2005) L2L: a simple tool for discovering the hidden significance in microarray expression data. Genome Biol 6: R81.
68. Cahan P, Ahmad A M, Burke H, Fu S, Lai Y, et al. (2005) List of lists-annotated (LOLA): a database for annotation and comparison of published microarray gene lists. Gene 360: 78-82.
69. Culhane A C, Schwarzl T, Sultana R, Picard K C, Picard S C, et al. (2010) GeneSigDB—a curated database of gene expression signatures. Nucleic Acids Res 38: D716-D725.
70. Rhodes D R, Barrette T R, Rubin M A, Ghosh D, and Chinnaiyan A M. (2002) Meta-analysis of microarrays: interstudy validation of gene expression profiles reveals pathway dysregulation in prostate cancer. Cancer Res 62: 4427-4433.
71. Lamb J, Crawford E D, Peck D, Modell J W, Blat I C, et al. (2006) The Connectivity Map: using gene-expression signatures to connect small molecules, genes, and disease. Science 313: 1929-1935.
72. Fan C, Oh D S, Wessels L, Weigelt B, Nuyten D S, et al. (2006) Concordance among gene-expression-based predictors for breast cancer. N Engl J Med 355: 560-569.
73. Venet D, Dumont J E, and Detours V. (2011) Most random gene expression signatures are significantly associated with breast cancer outcome. PLoS Comput Biol 7: e1002240.
74. Haibe-Kains B, Desmedt C, Piette F, Buyse M, Cardoso F, et al. (2008) Comparison of prognostic gene expression signatures for breast cancer. BMC Genomics 9: 394.
75. Buyse M, Loi S, van't V L, Viale G, Delorenzi M, et al. (2006) Validation and clinical utility of a 70-gene prognostic signature for women with node-negative breast cancer. J Natl Cancer Inst 98: 1183-1192.
76. Bueno-de-Mesquita J M, Linn S C, Keijzer R, Wesseling J, Nuyten D S, et al. (2009) Validation of 70-gene prognosis signature in node-negative breast cancer. Breast Cancer Res Treat 117: 483-495.
77. Bueno-de-Mesquita J M, van Harten W H, Retel V P, van't Veer L J, van Dam F S, et al. (2007) Use of 70-gene signature to predict prognosis of patients with node-negative breast cancer: a prospective community-based feasibility study (RASTER). Lancet Oncol 8: 1079-1087.
78. Mook S, Schmidt M K, Viale G, Pruneri G, Eekhout I, et al. (2009) The 70-gene prognosis-signature predicts disease outcome in breast cancer patients with 1-3 positive lymph nodes in an independent validation study. Breast Cancer Res Treat 116: 295-302.
79. Cardoso F, van't V L, Rutgers E, Loi S, Mook S, et al. (2008) Clinical application of the 70-gene profile: the MINDACT trial. J Clin Oncol 26: 729-735.
80. Strayer M E, Glas A M, Hannemann J, Wesseling J, van d, V, et al. (2010) The 70-gene signature as a response predictor for neoadjuvant chemotherapy in breast cancer. Breast Cancer Res Treat 119: 551-558.
81. Foekens J A, Atkins D, Zhang Y, Sweep F C, Harbeck N, et al. (2006) Multicenter validation of a gene expression-based prognostic signature in lymph node-negative primary breast cancer. J Clin Oncol 24: 1665-1671.
82. McDermott U, Downing J R, and Stratton M R. (2011) Genomics and the continuum of cancer care. N Engl J Med 364: 340-350.
83. Geradts J, Bean S M, Bentley R C, and Barry W T. (2010) The oncotype DX recurrence score is correlated with a composite index including routinely reported pathobiologic features. Cancer Invest 28: 969-977.
84. Ntzani E E and Ioannidis J P. (2003) Predictive ability of DNA microarrays for cancer outcomes and correlates: an empirical assessment. Lancet 362: 1439-1444.
85. Michiels S, Koscielny S, and Hill C. (2005) Prediction of cancer outcome with microarrays: a multiple random validation strategy. Lancet 365: 488-492.
86. Ein-Dor L, Kela I, Getz G, Givol D, and Domany E. (2005) Outcome signature genes in breast cancer: is there a unique set? Bioinformatics 21: 171-178.
87. Lin Y H, Friederichs J, Black M A, Mages J, Rosenberg R, et al. (2007) Multiple gene expression classifiers from different array platforms predict poor prognosis of colorectal cancer. Clin Cancer Res 13: 498-507.
88. Fan X, Shi L, Fang H, Cheng Y, Perkins R, et al. (2010) DNA microarrays are predictive of cancer prognosis: a re-evaluation. Clin Cancer Res 16: 629-636.
89. Haibe-Kains B, Desmedt C, Loi S, Culhane A C, Bontempi G, et al. (2012) A three-gene model to robustly identify breast cancer molecular subtypes. J Natl Cancer Inst 104: 311-325.
90. Fan C, Prat A, Parker J S, Liu Y, Carey L A, et al. (2011) Building prognostic models for breast cancer patients using clinical variables and hundreds of gene expression signatures. BMC Med Genomics 4: 3.
91. Desmedt C, Haibe-Kains B, Wirapati P, Buyse M, Larsimont D, et al. (2008) Biological processes associated with breast cancer clinical outcome depend on the molecular subtypes. Clin Cancer Res 14: 5158-5165.
92. Mehta R, Jain R K, and Badve S. (2011) Personalized medicine: the road ahead. Clin Breast Cancer 11: 20-26.
93. Liu R, Wang X, Chen G Y, Dalerba P, Gurney A, et al. (2007) The prognostic role of a gene signature from tumorigenic breast-cancer cells. N Engl J Med 356: 217-226.
94. Prat A, Ellis M J, and Perou C M. (2012) Practical implications of gene-expression-based assays for breast oncologists. Nat Rev Clin Oncol 9: 48-57.

### SUPPLEMENTAL REFERENCES

1. Yi Y, Li C, Miller C, George A L, Jr. (2007) Strategy for encoding and comparison of gene expression signatures. Genome Biol 8: R133.
2. Rhodes D R, Barrette T R, Rubin M A, Ghosh D, Chinnaiyan A M (2002) Meta-analysis of microarrays: interstudy validation of gene expression profiles reveals pathway dysregulation in prostate cancer. Cancer Res 62: 4427-4433.
3. van d, V, He Y D, van't Veer L J, Dai H, Hart A A et al. (2002) A gene-expression signature as a predictor of survival in breast cancer. N Engl J Med 347: 1999-2009.
4. Loi S, Haibe-Kains B, Desmedt C, Wirapati P, Lallemand F et al. (2008) Predicting prognosis using molecular profiling in estrogen receptor-positive breast cancer treated with tamoxifen. BMC Genomics 9: 239.
5. Hu Z, Fan C, Oh D S, Marron J S, He X et al. (2006) The molecular portraits of breast tumors are conserved across microarray platforms. BMC Genomics 7: 96.
6. Nakamura Y, Tanaka F, Haraguchi N, Mimori K, Matsumoto T et al. (2007) Clinicopathological and biological significance of mitotic centromere-associated kinesin overexpression in human gastric cancer. Br J Cancer 97: 543-549.
7. Li G Q, Li H, Zhang H F (2003) Mad2 and p53 expression profiles in colorectal cancer and its clinical significance. World J Gastroenterol 9: 1972-1975.
8. Fluge O, Gravdal K, Carlsen E, Vonen B, Kjellevold K et al. (2009) Expression of EZH2 and Ki-67 in colorectal cancer and associations with treatment response and prognosis. Br J Cancer 101: 1282-1289.
9. Samaras V, Stamatelli A, Samaras E, Arnaoutoglou C, Arnaoutoglou M et al. (2009)
Comparative immunohistochemical analysis of aurora-A and aurora-B expression in human glioblastomas. Associations with proliferative activity and clinicopathological features. Pathol Res Pract 205: 765-773.
10. de R A, Assie G, Rickman D S, Tissier F, Groussin L et al. (2009) Gene expression profiling reveals a new classification of adrenocortical tumors and identifies molecular predictors of malignancy and survival. J Clin Oncol 27: 1108-1115.
11. Chen M F, Lee K D, Lu M S, Chen C C, Hsieh M J et al. (2009) The predictive role of E2-EPF ubiquitin carrier protein in esophageal squamous cell carcinoma. J Mol Med 87: 307-320.
12. Petropoulou C, Kotantaki P, Karamitros D, Taraviras S (2008) Cdt1 and Geminin in cancer: markers or triggers of malignant transformation? Front Biosci 13: 4485-4494.
13. Dankof A, Fritzsche F R, Dahl E, Pahl S, Wild P et al. (2007) KPNA2 protein expression in invasive breast carcinoma and matched peritumoral ductal carcinoma in situ. Virchows Arch 451: 877-881.
14. Miyashita M, Yoshimura H, Hatta K, Tachibana S, Kubota M et al. (2009) [Clinical significance of intratumoral TS levels and DPD activity in breast cancer]. Gan To Kagaku Ryoho 36: 407-411.
15. Kadara H, Lacroix L, Behrens C, Solis L, Gu X et al. (2009) Identification of gene signatures and molecular markers for human lung cancer prognosis using an in vitro lung carcinogenesis system. Cancer Prev Res (Phila Pa) 2: 702-711.
16. Cooper W A, Kohonen-Corish M R, McCaughan B, Kennedy C, Sutherland R L et al. (2009) Expression and prognostic significance of cyclin B1 and cyclin A in non-small cell lung cancer. Histopathology 55: 28-36.
17. Boukovinas I, Papadaki C, Mendez P, Taron M, Mavroudis D et al. (2008) Tumor BRCA1, RRM1 and RRM2 mRNA expression levels and clinical response to first-line gemcitabine plus docetaxel in non-small-cell lung cancer patients. PLoS One 3: e3695.
18. Stay D, Bar I, Sandbank J (2007) Usefulness of CDK5RAP3, CCNB2, and RAGE genes for the diagnosis of lung adenocarcinoma. Int J Biol Markers 22: 108-113.
19. Glinsky G V, Berezovska O, Glinskii A B (2005) Microarray analysis identifies a death-from-cancer signature predicting therapy failure in patients with multiple types of cancer. J Clin Invest 115: 1503-1521.
20. Lee H, Kim D, Dan H C, Wu E L, Gritsko T M et al. (2007) Identification and characterization of putative tumor suppressor NGB, a GTP-binding protein that interacts with the neurofibromatosis 2 protein. Mol Cell Biol 27: 2103-2119.
21. Karmakar S, Foster E A, Smith C L (2009) Estradiol downregulation of the tumor suppressor gene BTG2 requires estrogen receptor-alpha and the REA corepressor. Int J Cancer 124: 1841-1851.
22. Cheng C J, Lin Y C, Tsai M T, Chen C S, Hsieh M C et al. (2009) SCUBE2 suppresses breast tumor cell proliferation and confers a favorable prognosis in invasive breast cancer. Cancer Res 69: 3634-3641.
23. Wu J, Qiu Q, Xie L, Fullerton J, Yu J et al. (2009) Web-based interrogation of gene expression signatures using EXALT. BMC Bioinformatics 10: 420.
24. Newman J C, Weiner A M (2005) L2L: a simple tool for discovering the hidden significance in microarray expression data. Genome Biol 6: R81.
25. Cahan P, Ahmad A M, Burke H, Fu S, Lai Y et al. (2005) List of lists-annotated (LOLA): a database for annotation and comparison of published microarray gene lists. Gene 360: 78-82.
26. Culhane A C, Schwarzl T, Sultana R, Picard K C, Picard S C et al. (2010) GeneSigDB—a curated database of gene expression signatures. Nucleic Acids Res 38: D716-D725.
27. Rhodes D R, Barrette T R, Rubin M A, Ghosh D, Chinnaiyan A M (2002) Meta-analysis of microarrays: interstudy validation of gene expression profiles reveals pathway dysregulation in prostate cancer. Cancer Res 62: 4427-4433.
28. Lamb J, Crawford E D, Peck D, Modell J W, Blat I C et al. (2006) The Connectivity Map: using gene-expression signatures to connect small molecules, genes, and disease. Science 313: 1929-1935.
29. Subramanian A, Tamayo P, Mootha V K, Mukherjee S, Ebert B L et al. (2005) Gene set enrichment analysis: a knowledge-based approach for interpreting genome-wide expression profiles. Proc Natl Acad Sci USA 102: 15545-15550.
30. Rhodes D R, Yu J, Shanker K, Deshpande N, Varambally R et al. (2004) Large-scale meta-analysis of cancer microarray data identifies common transcriptional profiles of neoplastic transformation and progression. Proc Natl Acad Sci USA 101: 9309-9314.
31. Chen S, Chen Y, Hu C, Jing H, Cao Y et al. (2010) Association of clinicopathological features with UbcH10 expression in colorectal cancer. J Cancer Res Clin Oncol 136: 419-426.
32. Jiang L, Bao Y, Luo C, Hu G, Huang C et al. (2010) Knockdown of ubiquitin-conjugating enzyme E2C/UbcH10 expression by RNA interference inhibits glioma cell proliferation and enhances cell apoptosis in vitro. J Cancer Res Clin Oncol 136: 211-217.
33. Shimo A, Tanikawa C, Nishidate T, Lin M L, Matsuda K et al. (2008) Involvement of kinesin family member 2C/mitotic centromere-associated kinesin overexpression in mammary carcinogenesis. Cancer Sci 99: 62-70.
34. Yim E K, Tong S Y, Ho E M, Bae J H, Um S J et al. (2009) Anticancer effects on TACC3 by treatment of paclitaxel in HPV-18 positive cervical carcinoma cells. Oncol Rep 21: 549-557.
35. Zhang S H, Xu A M, Chen X F, Li D H, Sun M P et al. (2008) Clinicopathologic significance of mitotic arrest defective protein 2 overexpression in hepatocellular carcinoma. Hum Pathol 39: 1827-1834.
36. Shang X, Burlingame S M, Okcu M F, Ge N, Russell H V et al. (2009) Aurora A is a negative prognostic factor and a new therapeutic target in human neuroblastoma. Mol Cancer Ther 8: 2461-2469.
37. Inoda S, Hirohashi Y, Torigoe T, Nakatsugawa M, Kiriyama K et al. (2009) Cep55/c10orf3, a tumor antigen derived from a centrosome residing protein in breast carcinoma. J Immunother 32: 474-485.
38. Chen C H, Chien C Y, Huang C C, Hwang C F, Chuang H C et al. (2009) Expression of FLJ10540 is correlated with aggressiveness of oral cavity squamous cell carcinoma by stimulating cell migration and invasion through increased FOXM1 and MMP-2 activity. Oncogene 28: 2723-2737.
39. Zheng H, Hu W, Deavers M T, Shen D Y, Fu S et al. (2009) Nuclear cyclin B1 is overexpressed in low-malignant-potential ovarian tumors but not in epithelial ovarian cancer. Am J Obstet Gynecol 201: 367-6.
40. de H T, Hasselt N, Troost D, Caron H, Popovic M et al. (2008) Molecular risk stratification of medulloblastoma patients based on immunohistochemical analysis of MYC, LDHB, and CCNB1 expression. Clin Cancer Res 14: 4154-4160.
41. Zhang K, Hu S, Wu J, Chen L, Lu J et al. (2009) Overexpression of RRM2 decreases thrombspondin-1 and increases VEGF production in human cancer cells in vitro and in vivo: implication of RRM2 in angiogenesis. Mol Cancer 8: 11.
42. Duxbury M S, Ito H, Zinner M J, Ashley S W, Whang E E (2004) RNA interference targeting the M2 subunit of ribonucleotide reductase enhances pancreatic adenocarcinoma chemosensitivity to gemcitabine. Oncogene 23: 1539-1548.
43. Zhao L, Qin L X, Ye Q H, Zhu X Q, Zhang H et al. (2004) KIAA0008 gene is associated with invasive phenotype of human hepatocellular carcinoma—a functional analysis. J Cancer Res Clin Oncol 130: 719-727.
44. Szponar A, Zubakov D, Pawlak J, Jauch A, Kovacs G (2009) Three genetic developmental stages of papillary renal cell tumors: duplication of chromosome 1q marks fatal progression. Int J Cancer 124: 2071-2076.
45. Tsunoda N, Kokuryo T, Oda K, Senga T, Yokoyama Y et al. (2009) Nek2 as a novel molecular target for the treatment of breast carcinoma. Cancer Sci 100: 111-116.
46. Xiao G F, Tang H H (2008) [Expression and clinical significance of highly expressed protein in cancer (Hec 1) in human primary gallbladder carcinoma]. Xi Bao Yu Fen Zi Mian Yi Xue Za Zhi 24: 910-912.
47. Park S H, Yu G R, Kim W H, Moon W S, Kim J H et al. (2007) NF-Y-dependent cyclin B2 expression in colorectal adenocarcinoma. Clin Cancer Res 13: 858-867.
48. Taniuchi K, Nakagawa H, Nakamura T, Eguchi H, Ohigashi H et al. (2005) Downregulation of RAB6KIFL/KIF20A, a kinesin involved with membrane trafficking of discs large homologue 5, can attenuate growth of pancreatic cancer cell. Cancer Res 65: 105-112.
49. Kang J U, Koo S H, Kwon K C, Park J W, Kim J M (2008) Gain at chromosomal region 5p15.33, containing TERT, is the most frequent genetic event in early stages of non-small cell lung cancer. Cancer Genet Cytogenet 182: 1-11.
50. Jiang R, Xia Y, Li J, Deng L, Zhao L et al. (2010) High expression levels of IKKalpha and IKKbeta are necessary for the malignant properties of liver cancer. Int J Cancer 126: 1263-1274.
51. Mitra A, Jameson C, Barbachano Y, Sanchez L, Kote-Jarai Z et al. (2009) Overexpression of RAD51 occurs in aggressive prostatic cancer. Histopathology 55: 696-704.
52. Naoe M, Ogawa Y, Morita J, Shichijo T, Fuji K et al. (2009) Expression of the fluoropyrimidine-metabolizing enzymes in bladder cancers as measured by the Danenberg tumor profile. Oncol Res 18: 153-162.
53. Seo J, Chung Y S, Sharma G G, Moon E, Burack W R et al. (2005) Cdt1 transgenic mice develop lymphoblastic lymphoma in the absence of p53. Oncogene 24: 8176-8186.
54. Singh P, Yang M, Dai H, Yu D, Huang Q et al. (2008) Overexpression and hypomethylation of flap endonuclease 1 gene in breast and other cancers. Mol Cancer Res 6: 1710-1717.
55. Arai M, Kondoh N, Imazeki N, Hada A, Hatsuse K et al. (2009) The knockdown of endogenous replication factor C4 decreases the growth and enhances the chemosensitivity of hepatocellular carcinoma cells. Liver Int 29: 55-62.
56. Karanikolas B D, Figueiredo M L, Wu L (2010) Comprehensive evaluation of the role of EZH2 in the growth, invasion, and aggression of a panel of prostate cancer cell lines. Prostate 70: 675-688.
57. Sugiura T, Nagano Y, Noguchi Y (2007) DDX39, upregulated in lung squamous cell cancer, displays RNA helicase activities and promotes cancer cell growth. Cancer Biol Ther 6: 957-964.
58. Ooe A, Kato K, Noguchi S (2007) Possible involvement of CCT5, RGS3, and YKT6 genes up-regulated in p53-mutated tumors in resistance to docetaxel in human breast cancers. Breast Cancer Res Treat 101: 305-315.
59. Wang Y, Ma Y, Lu B, Xu E, Huang Q et al. (2007) Differential expression of mimecan and thioredoxin domain-containing protein 5 in colorectal adenoma and cancer: a proteomic study. Exp Biol Med (Maywood)) 232: 1152-1159.
60. Majid S M, Liss A S, You M, Bose H R (2006) The suppression of SH3BGRL is important for v-Rel-mediated transformation. Oncogene 25: 756-768.
61. Curtis C, Shah S P, Chin S F, Turashvili G, Rueda O M et al. (2012) The genomic and transcriptomic architecture of 2,000 breast tumours reveals novel subgroups. Nature 486: 346-352.
62. van', V, Dai H, van d, V, He Y D, Hart A A et al. (2002) Gene expression profiling predicts clinical outcome of breast cancer. Nature 415: 530-536.
63. Wang Y, Klijn J G, Zhang Y, Sieuwerts A M, Look M P et al. (2005) Gene-expression profiles to predict distant metastasis of lymph-node-negative primary breast cancer. Lancet 365: 671-679.
64. Paik S, Shak S, Tang G, Kim C, Baker J et al. (2004) A multigene assay to predict recurrence of tamoxifen-treated, node-negative breast cancer. N Engl J Med 351: 2817-2826.
65. Flanagan M B, Dabbs D J, Brufsky A M, Beriwal S, Bhargava R (2008) Histopathologic variables predict Oncotype DX recurrence score. Mod Pathol 21: 1255-1261.
66. Parker J S, Mullins M, Cheang M C, Leung S, Voduc D et al. (2009) Supervised risk predictor of breast cancer based on intrinsic subtypes. J Clin Oncol 27: 1160-1167.
67. Haibe-Kains B, Desmedt C, Rothe F, Piccart M, Sotiriou C et al. (2010) A fuzzy gene expression-based computational approach improves breast cancer prognostication. Genome Biol 11: R18.
68. Loi S, Haibe-Kains B, Majjaj S, Lallemand F, Durbecq V et al. (2010) PIK3CA mutations associated with gene signature of low mTORC1 signaling and better outcomes in estrogen receptor-positive breast cancer. Proc Natl Acad Sci USA 107: 10208-10213.
69. Sotiriou C, Wirapati P, Loi S, Harris A, Fox S et al. (2006) Gene expression profiling in breast cancer: understanding the molecular basis of histologic grade to improve prognosis. J Natl Cancer Inst 98: 262-272.
70. Qiu, Q, Lu, P., Xiang, Y., Shyr, Y., Chen, X., Lehmann, B. D., Viox, D. J., George, A. L., Jr., Yi, Y. (2013) A Data Similarity-Based Stategy for Meta-analysis of Transcriptional Profiles in Cancer, PLOS ONE, 8(1): es4979, pp. 1-13.

It will be understood that various details of the presently disclosed subject matter can be changed without departing from the scope of the subject matter disclosed herein. Furthermore, the foregoing description is for the purpose of illustration only, and not for the purpose of limitation.

TABLE 1
Members of Breast Cancer Metastatic Signature (BRmet50)

| Signature ID | Signature Name | BRid* |
|---|---|---|
| Sig544 | without metastasis vs with metastasis | BR544 [3] |
| Sig2411 | without metastasis vs with metastasis | BR2411 [11] |
| Sig1405 | ER-positive vs ER-negative | BR1405 [2] |
| Sig1128 | grade 1 vs grade 3 | BR1128 [24] |
| Sig1042 | grade 1 vs grade 3 | BR1042 [8] |
| Sig1224r | ER-positive vs ER-negative | BR1224 [43] |
| Sig1552r | normal breast-like vs basal-like | BR1552 [34] |
| Sig1095 | grade 1 vs grade 3 | BR1095 [25] |
| Sig1414 | grade 1 vs grade 3 | BR1414 [25] |
| Sig1141 | grade 1 vs grade 3 | BR1141 [6] |
| Sig907r | normal breast-like vs basal-like | BR907 [44] |

*BRid denotes the breast cancer dataset ID sharing the same signature ID number as the respective published study.

TABLE 2
Summary of survival analysis p-values in breast cancer

| Test Data Sets | Endpoints* | BRmet50 | BRmet50 Ctr** | BRSig70 | BRSig76 |
|---|---|---|---|---|---|
| Training datasets | | | | | |
| BR544 [3] | DMFS | <0.001 | <0.001 | 0.007 | 0.024 |
| BR2411 [11] | RFS | <0.001 | <0.001 | <0.001 | <0.001 |
| BR1405 [2] | RFS | 0.002 | 0.002 | 0.019 | 0.006 |
| BR1128 [24] | DSS | <0.001 | <0.001 | 0.015 | 0.018 |
| BR1042 [8] | RFS | 0.002 | 0.033 | 0.144 | 0.698 |
| BR1552 [34] | RFS | <0.001 | <0.001 | 0.082 | <0.001 |
| BR1095 [25] | DFS | <0.001 | <0.001 | 0.001 | 0.005 |
| BR1414 [25] | RFS | <0.001 | <0.001 | <0.001 | <0.001 |
| BR1141 [6] | RFS | <0.001 | <0.001 | 0.026 | 0.156 |
| BR18347175 [35] | DMFS | <0.001 | NA*** | <0.001 | <0.001 |
| Validation datasets | | | | | |
| METABRIC discovery [36] | DSS | <0.001 | NA | <0.001 | <0.001 |
| METABRIC validation [36] | DSS | <0.001 | NA | <0.001 | <0.001 |
| GSE2607 [33] | RFS | 0.004 | NA | 0.005 | <0.001 |
| GSE7390 [32] | RFS | 0.028 | NA | 0.516 | 0.063 |
| GSE11121 [31] | DMFS | 0.027 | NA | 0.012 | 0.183 |
| GSE17705 [29] | DMFS | 0.045 | NA | 0.043 | 0.574 |
| GSE20624 [28] | RFS | 0.001 | NA | 0.037 | 0.037 |
| GSE20685 [27] | OS | <0.001 | NA | 0.002 | <0.001 |
| GSE21653 [30] | DFS | 0.014 | NA | 0.121 | 0.396 |
| GSE25055 [26] | DMFS | <0.001 | NA | <0.001 | <0.001 |
| GSE25065 [26] | DMFS | <0.001 | NA | <0.001 | <0.001 |

*Endpoints: Clinic endpoints are distant metastases-free survival (DMFS), relapse-free survival (RFS), disease-free survival (DFS), disease-specific survival (DSS), Overall Survival (OS).
**BRmet50 Ctr: control signatures are isoform signatures of BRmet50 assembled by the leave-one-out method in which the corresponding breast cancer dataset is excluded intentionally.
***NA: not available.

TABLE 3
Hazard ratio risks for cancer relapse and log-rank tests in BR1141

| Clinicopathologic | BRmet50 | | BRsig70 | | BRsig76 | |
|---|---|---|---|---|---|---|
| parameters | HR (95% CI) | HR P | HR (95% CI) | HR P | HR (95% CI) | HR P |
| Tumor size | | | | | | |
| T1 | 2.6 (1.3-5.5) | 0.009 | 1.5 (0.6-3.7) | 0.386 | 1.0 (0.5-2.1) | 0.942 |
| T2 | 1.7 (1.0-2.8) | 0.044 | 1.8 (0.9-3.8) | 0.113 | 0.7 (0.4-1.2) | 0.209 |
| Lymph-node | | | | | | |
| involvement | | | | | | |
| No | 2.3 (1.4-3.9) | 0.001 | 1.6 (0.8-3.0) | 0.193 | 0.8 (0.5-1.4) | 0.511 |
| Yes | 2.0 (1.0-4.1) | 0.053 | 2.8 (0.8-9.3) | 0.089 | 0.6 (0.3-1.4) | 0.245 |
| Tamoxifen treatment | | | | | | |
| No | 2.6 (1.4-5.0) | 0.004 | 2.1 (1.0-4.6) | 0.063 | 1.1 (0.5-2.0) | 0.869 |
| Yes | 2.2 (1.2-3.9) | 0.007 | 1.7 (0.7-4.0) | 0.230 | 0.6 (0.3-1.0) | 0.041 |
| Differentiation | | | | | | |
| Good | 2.3 (0.6-8.4) | 0.196 | 2.4 (0.8-7.2) | 0.121 | 1.3 (0.4-3.8) | 0.682 |
| Intermediate | 2.5 (1.5-4.3) | 0.001 | 1.6 (0.8-3.4) | 0.219 | 0.7 (0.4-1.2) | 0.194 |
| Poor | 1.4 (0.6-3.4) | 0.442 | 0.2 (0-1.8) | 0.172 | 0.5 (0.2-1.1) | 0.086 |
| ER status | | | | | | |
| Negative | 1.4 (0.5-4.0) | 0.495 | 2.2 (0.3-16.3) | 0.456 | 0.9 (0.3-2.3) | 0.782 |
| Positive | 2.5 (1.6-4.0) | <0.001 | 1.8 (1.0-3.3) | 0.050 | 0.7 (0.4-1.1) | 0.103 |

The 269 patients with breast cancer included in the BR1141 dataset were stratified according to tumor size, lymph-node status, tamoxifen treatment, histological grade, and ER status. A univariate Cox proportional-hazards model was used to evaluate the association of individual signatures (i.e., the BRmet50, BRsig70, or BRsig76) with the clinical outcome in each category.
T1 denotes a tumor with size less than or equal to 2.0 cm, and T2 denotes a tumor with size larger than 2.0 cm. HR (95% CI): hazard ratio value (95% confidence interval).
HR P: hazard ratio p-value.

TABLE 4
Multivariate analysis of disease risk among patients with breast cancer

| | BRmet50 | | BRsig70 | | BRsig76 | |
|---|---|---|---|---|---|---|
| | HR | | HR | | HR | |
| Datasets | (95% CI) | HR P | (95% CI) | HR P | (95% CI) | HR P |
| BR1042 | 3.1 | <0.01 | 1.7 (0.7-3.9) | 0.23 | 0.8 (0.4-1.7) | 0.54 |
| | (1.4-7.0) | | | | | |
| BR1095 | 1.8 | 0.02 | 1.5 (0.9-2.5) | 0.16 | 1.3 (0.8-2.2) | 0.26 |
| | (1.1-2.9) | | | | | |
| BR1128 | 2.0 | 0.03 | 1.4 (0.8-2.7) | 0.27 | 1.2 (0.6-2.3) | 0.49 |
| | (1.0-3.9) | | | | | |
| BR1141 | 2.3 | <0.01 | 1.6 (0.9-2.9) | 0.13 | 0.6 (0.4-1.0) | 0.05 |
| | (1.4-3.6) | | | | | |
| GSE7390 | 2.5 | <0.01 | 1.1 (0.6-2.0) | 0.76 | 2.0 (1.1-3.3) | 0.03 |
| | (1.4-5.0) | | | | | |

The HR and p-values for each signature were adjusted by age, grade, tumor size, LN, ER, and NPI.
Age and tumor diameter were modeled as continuous variables; the hazard ratio is for each increase of 1 cm. in diameter or for each 1-year increase in age.
HR: hazard ratio with 95% confidence interval;
HR P: hazard ratio p-value.

TABLE 5
Univariate and multivariate analysis in lung, prostate, and colon cancer

| | | BRmet50 | | BRsig70 | | BRsig76 | |
|---|---|---|---|---|---|---|---|
| Cancer type | Analysis | HR (95% CI) | HR P | HR (95% CI) | HR P | HR (95% CI) | HR P |
| Lung [50] | Univariate | 1.7 (1.3-2.4) | <0.01 | 1.2 (0.8-1.9) | 0.37 | 1.2 (0.8-1.9) | 0.37 |
| | Multivariate* | 1.8 (1.2-2.5) | <0.01 | 1.2 (0.7-1.9) | 0.51 | 1.2 (0.7-1.9) | 0.51 |
| Prostate [51] | Univariate | 0.4 (0.3-0.6) | <0.01 | 1.2 (0.8-1.8) | 0.34 | 0.7 (0.5-1.0) | 0.07 |
| | Multivariate** | 0.6 (0.3-1.0) | 0.04 | 1.3 (0.8-2.1) | 0.27 | 0.9 (0.5-1.4) | 0.52 |
| Colon [49] | Univariate | 1.3 (0.4-4.7) | 0.66 | 0.5 (0.1-1.9) | 0.32 | 0.5 (0.2-1.9) | 0.32 |
| | Multivariate*** | 1.4 (0.4-5.0) | 0.62 | 0.5 (0.1-1.8) | 0.31 | 0.5 (0.1-1.8) | 0.30 |

*Adjusted factors in lung cancer: age, gender, chemotherapy treatment, radiation treatment, smoking habits, and tumor stage.
**Adjusted factors in prostate cancer: age, tumor stage, ploidy, and PSA relapse.
***Adjusted factor in colon cancer: age.

TABLE S1
Breast cancer dataset source and derived signature phenotypes

| Study Name | PMID | Signature Phenotypes |
|---|---|---|
| Adler AS et al Nat Genet 2006 | 16518402 | CSN5, GFP, MYC, and MYC + CSN5 |
| Anders CK et al PLoS ONE 2008 | 18167534 | age comparison ranging from 45 to 65 years old |
| Anders CK et al PLoS ONE 2008 | 18167534 | ductal NOS and infiltrating duct lobular carcinoma |
| Anders CK et al PLoS ONE 2008 | 18167534 | ER negative vs. ER positive |
| Anders CK et al PLoS ONE 2008 | 18167534 | mono-, non-, and tri-chemotherapy |
| Anders CK et al PLoS ONE 2008 | 18167534 | node negative vs. node positive |
| Anders CK et al PLoS ONE 2008 | 18167534 | nuclear grade 1, 2, and 3 |
| Anders CK et al PLoS ONE 2008 | 18167534 | pathological stages 1, 2A, 2B, and 3B |
| Anders CK et al PLoS ONE 2008 | 18167534 | PR negative vs. PR positive |
| Anders CK et al PLoS ONE 2008 | 18167534 | recurrence vs. no recurrence |
| Anders CK et al PLoS ONE 2008 | 18167534 | TNM stages 1A, 1B, 1C, and 2 |
| Anders CK et al PLoS ONE 2008 | 18167534 | tumor size comparison between 2.0 cm to 5.0 cm |
| Anders CK et al PLoS ONE 2008 | 18167534 | undifferentiated, moderately, and poorly differentiated |
| Anders CK et al PLoS ONE 2008 | 18167534 | without hormone therapy vs. with hormone therapy |
| ang E et al Lancet 2003 | 12747878 | lymph node status in breast cancer |
| ang E et al Lancet 2003 | 12747878 | relapse status in breast cancer |
| ardson AL et al Cancer Cell 2006 | 16473279 | BRCA mutation negative vs. BRCA mutation positive |
| ardson AL et al Cancer Cell 2006 | 16473279 | ER negative vs. ER positive |
| ardson AL et al Cancer Cell 2006 | 16473279 | HER2 negative vs. HER2 positive |
| ardson AL et al Cancer Cell 2006 | 16473279 | PR negative vs. PR positive |
| Bhati R et al Am J Pathol 2008 | 18403594 | vascular cells from breast tumor samples |
| Bild AH et al Nature 2006 | 16273092 | Bcat1-9, E2F3 1-10, RAS1-10, GFP1-10, Myc1-10, and |
| | | Src1-7 |
| Bild AH et al Nature 2006 | 16273092 | ER status levels 0 1 vs. ER status level 2 3 |
| Bild AH et al Nature 2006 | 16273092 | ER status level 0 vs. ER status levels 1 2 3 |
| Bild AH et al Nature 2006 | 16273092 | squamous and adenocarcinoma |
| Chanrion M et al Clin Cancer Res | 18347175 | ER negative vs. ER positive |
| 2008 | | |
| Chanrion M et al Clin Cancer Res | 18347175 | grades 1, 2, and 3 |
| 2008 | | |
| Chanrion M et al Clin Cancer Res | 18347175 | no distant metastasis vs. distant metastasis |
| 2008 | | |
| Chanrion M et al Clin Cancer Res | 18347175 | no local recurrence vs. local recurrence |
| 2008 | | |
| Chanrion M et al Clin Cancer Res | 18347175 | node negative vs. node positive |
| 2008 | | |
| Chanrion M et al Clin Cancer Res | 18347175 | PR negative vs. PR positive |
| 2008 | | |
| Chanrion M et al Clin Cancer Res | 18347175 | x-ray and Tam, Tam, x-ray Tam and LHR |
| 2008 | | |
| Chapman SC et al BMC Dev Biol | 17663788 | node status and subtypes |
| 2007 | | |
| Chi JT et al PLoS Genet 2007 | 17907811 | smooth muscle tissue set 1 and breast cancer |
| | | prognosis |
| Chi JT et al PLoS Genet 2007 | 17907811 | smooth muscle tissue set 2 and breast cancer |
| | | porgnosis |
| Chi JT et al PLoS Genet 2007 | 17907811 | smooth muscle tissue set 3 and breast cancer |
| | | prognosis |
| Cicatiello L et al J Mol Endocrinol | 15171711 | estrogen actions in breast cancer cells from 1 h to 32 h |
| 2004 | | |
| Cicatiello L et al J Mol Endocrinol | 15171711 | sham-treated p53 RNAi and Dox-treated cell lines |
| 2004 | | |
| Dairkee SH et al BMC Genomics | 15260889 | mammary epithelial tumor tissue and breast cancer cell |
| 2004 | | lines |
| Dittmer A et al J Biol Chem 2006 | 16551631 | cells, transfected with siPTHrP vs. control siRNA |
| er WR et al Pharmacogent | | |
| Genomics 2007 | 17885619 | breast tumor with letrozole treatmentl for 10 to 14 days |
| Farmer P et al Oncogene 2005 | 15897907 | AR negative vs. AR positive |
| Farmer P et al Oncogene 2005 | 15897907 | ER negative vs. ER positive |
| Finak G et al Breast Cancer Res | 17054791 | ER status |
| 2006 | | |
| Finak G et al Breast Cancer Res | 17054791 | grades 2 and 3 |
| 2006 | | |
| Finak G et al Breast Cancer Res | 17054791 | HER2 status |
| 2006 | | |
| Finak G et al Breast Cancer Res | 17054791 | node status |
| 2006 | | |
| Finak G et al Breast Cancer Res | 17054791 | normal epithelium and stroma adjacent to I.D.C. |
| 2006 | | |
| Finak G et al Breast Cancer Res | 17054791 | post-, pre-, and surgical menopause |
| 2006 | | |
| Finak G et al Nat Med 2008 | 18438415 | ER positive vs. ER negative |
| Finak G et al Nat Med 2008 | 18438415 | ERBB2 positive vs. ERBB2 negative |
| Finak G et al Nat Med 2008 | 18438415 | grade 1 vs. grade 2 |
| Finak G et al Nat Med 2008 | 18438415 | node positive vs. Node negative |
| Finak G et al Nat Med 2008 | 18438415 | PR positive vs. PR negative |
| Finak G et al Nat Med 2008 | 18438415 | tumor stroma vs. matched normal stroma |
| Foekens JA et al J Clin Oncol 2006 | 16505412 | highly, morderately, and poorly differentiated tumors |
| Foekens JA et al J Clin Oncol 2006 | 16505412 | with recurrence vs. without recurrence |
| Gruvberger et al Clin Cancer Res | 17404078 | ER alpha negative vs. ER alpha positive |
| 2007 | | |
| Hegde PS et al Mol Cancer Ther | 17513611 | Latinib treatment in time series |
| 2007 | | |
| Hernandez-V H et al Int J Cancer | 16557594 | MCF7 treated by 5-FU in time series |
| 2006 | | |
| Herschkowitz JI et al Breast Cancer | 18782450 | breast Cancer subtypes |
| Res 2008 using GPL1390 | | |
| Herschkowitz JI et al Breast Cancer | 18782450 | grades 1, 2, and 3 |
| Res 2008 using GPL1390 | | |
| Herschkowitz JI et al Breast Cancer | 18782450 | less than 45, 45 to 65, more than 65 |
| Res 2008 using GPL1390 | | |
| Herschkowitz JI et al Breast Cancer | 18782450 | lymph node status |
| Res 2008 using GPL1390 | | |
| Herschkowitz JI et al Breast Cancer | 18782450 | no relapse vs. relapse or die of disease-related death |
| Res 2008 using GPL1390 | | |
| Herschkowitz JI et al Breast Cancer | 18782450 | tumor size comparison and local invasion |
| Res 2008 using GPL1390 | | |
| Herschkowitz JI et al Breast Cancer | 18782450 | breast cancer subtypes |
| Res 2008 using GPL885 | | |
| Herschkowitz JI et al Breast Cancer | 18782450 | metastasis vs. no metastasis |
| Res 2008 using GPL885 | | |
| Herschkowitz JI et al Breast Cancer | 18782450 | breast cancer subtypes |
| Res 2008 using GPL887 | | |
| Herschkowitz JI et al Breast Cancer | 18782450 | ER negative vs. ER positive |
| Res 2008 using GPL887 | | |
| Herschkowitz JI et al Breast Cancer | 18782450 | grade 1, 2, and 3 |
| Res 2008 using GPL887 | | |
| Herschkowitz JI et al Breast Cancer | 18782450 | no relapse vs. relapse or die of disease |
| Res 2008 using GPL887 | | |
| Herschkowitz JI et al Breast Cancer | 18782450 | tumor sizes and direct extension to chest wall or skin |
| Res 2008 using GPL887 | | |
| Herschkowitz JI et al Breast Cancer | 18782450 | ER negative vs. ER positive |
| Res 2008 using GPL1390 | | |
| Herschkowitz JI et al Breast Cancer | 18782450 | 45 to 65 vs. older than 65 |
| Res 2008 using GPL885 | | |
| Herschkowitz JI et al Breast Cancer | 18782450 | ER negative vs. ER positive |
| Res 2008 using GPL885 | | |
| Herschkowitz JI et al Breast Cancer | 18782450 | younger than 45 vs. 45 to 65 |
| Res 2008 using GPL887 | | |
| Hoadley KA et al BMC Genomics | 17663798 | relapse and breast cancer subtypes |
| 2007 | | |
| Hoadley KA et al BMC Genomics | 17663798 | Control and treatment in time series |
| 2007 | | |
| Hoadley KA et al BMC Genomics | 17663798 | ER status and breast cancer subtypes |
| 2007 | | |
| Hoadley KA et al BMC Genomics | 17663798 | grade status and breast cancer subtypes |
| 2007 | | |
| Hu Z et al BMC Genomics 2006 | 16643655 | ER negative vs. ER positive |
| using GPL1390 | | |
| Hu Z et al BMC Genomics 2006 | 16643655 | node negative vs. node positive |
| using GPL1390 | | |
| Hu Z et al BMC Genomics 2006 | 16643655 | ER negative vs. ER positive |
| using GPL887 | | |
| Hu Z et al BMC Genomics 2006 | 16643655 | node negative vs. node positive |
| using GPL887 | | |
| Hu Z et al BMC Genomics 2006 | 16643655 | breast cancer subtypes |
| using GPL1390 | | |
| Hu Z et al BMC Genomics 2006 | 16643655 | grades 1, 2, and 3 |
| using GPL1390 | | |
| Hu Z et al BMC Genomics 2006 | 16643655 | no relapse vs. relapse |
| using GPL1390 | | |
| Hu Z et al BMC Genomics 2006 | 16643655 | ER negative and breast cancer subtypes |
| using GPL885 | | |
| Hu Z et al BMC Genomics 2006 | 16643655 | ER status and breast cancer subtypes |
| using GPL887 | | |
| Hu Z et al BMC Genomics 2006 | 16643655 | grades 1, 2, and 3 |
| using GPL887 | | |
| Hu Z et al BMC Genomics 2006 | 16643655 | no relapse vs. relapse |
| using GPL887 | | |
| ider J et al Int J Cancer 2006 | 17019712 | ER positive vs. ER negative |
| ider J et al Int J Cancer 2006 | 17019712 | PR positive vs. PR negative |
| Ivshina AV et al Cancer Res 2006 | 17079448 | luminal A, luminal B, Basal, ERBB2, and normal like |
| using GPL96 | | |
| Ivshina AV et al Cancer Res 2006 | 17079448 | 1-like, 2a-, 2b-, and 3-like |
| using GPL96 | | |
| Ivshina AV et al Cancer Res 2006 | 17079448 | ELSTON grades 1, 2, and 3 |
| using GPL96 | | |
| Ivshina AV et al Cancer Res 2006 | 17079448 | ER negative vs. ER positive |
| using GPL96 | | |
| Ivshina AV et al Cancer Res 2006 | 17079448 | no recurrence vs. recurrence |
| using GPL96 | | |
| Ivshina AV et al Cancer Res 2006 | 17079448 | node negative vs. node positive |
| using GPL96 | | |
| Ivshina AV et al Cancer Res 2006 | 17079448 | p53 wild type vs. p53 mutant |
| using GPL96 | | |
| Ivshina AV et al Cancer Res 2006 | 17079448 | relapse vs. no relapse |
| using GPL96 | | |
| Ivshina AV et al Cancer Res 2006 | 17079448 | 1-like, 2a-, 2b-, and 3-like |
| using GPL97 | | |
| Ivshina AV et al Cancer Res 2006 | 17079448 | ELSTON grades 1, 2, and 3 |
| using GPL97 | | |
| Ivshina AV et al Cancer Res 2006 | 17079448 | ER negative vs. ER positive |
| using GPL97 | | |
| Ivshina AV et al Cancer Res 2006 | 17079448 | luminal A, luminal B, Basal, ERBB2, and normal like |
| using GPL97 | | |
| Ivshina AV et al Cancer Res 2006 | 17079448 | no recurrence vs. recurrence |
| using GPL97 | | |
| Ivshina AV et al Cancer Res 2006 | 17079448 | node negative vs. node positive |
| using GPL97 | | |
| Ivshina AV et al Cancer Res 2006 | 17079448 | p53 wild type vs. p53 mutant |
| using GPL97 | | |
| Ivshina AV et al Cancer Res 2006 | 17079448 | relapse vs. no relapse |
| using GPL97 | | |
| Jones C et al Cancer Res 2004 | 15126339 | luminal breast cells and myoepithelial breast cells |
| Julka PK et al Br J Cancer 2008 | 18382427 | ER negative vs. ER positive |
| using GPL887 | | |
| Julka PK et al Br J Cancer 2008 | 18382427 | HER2 negative vs. HER2 positive |
| using GPL887 | | |
| Julka PK et al Br J Cancer 2008 | 18382427 | luminal A, luminal B, basal, HER2, and normal like |
| using GPL887 | | |
| Julka PK et al Br J Cancer 2008 | 18382427 | poorly differentiated vs. moderately differentiated |
| using GPL887 | | |
| Julka PK et al Br J Cancer 2008 | 18382427 | PR negative vs. PR positive |
| using GPL887 | | |
| Julka PK et al Br J Cancer 2008 | 18382427 | TNM stages T3 and T4 |
| using GPL887 | | |
| Kang Yet al Cancer Cell 2003 | 12842083 | control, bone metastasis, and adrenal gland metastasis |
| Kang Y et al Cancer Cell 2003 | 12842083 | highly metastasis phenotype vs. weakly metastasis |
| | | phenotype |
| Kreike B et al Clin Cancer Res 2006 | 17020974 | primary tumors without recurrence vs. recurrences |
| Lamb J et al Science 2006 | 17008526 | 17-allylamino-geldanamycin effects to breast cancer cell |
| | | lines |
| Lamb J et al Science 2006 | 17008526 | control and 17-allylamino-geldanamycin |
| Lamb J et al Science 2006 | 17008526 | control and 17-dimethylamino-geldanamycin |
| Lamb J et al Science 2006 | 17008526 | control and Alpha Estradiol |
| Lamb J et al Science 2006 | 17008526 | control and Arachidonic |
| Lamb J et al Science 2006 | 17008526 | control and Geldanamycin |
| Lamb J et al Science 2006 | 17008526 | control vs. Haloperidol |
| Lamb J et al Science 2006 | 17008526 | control vs. thioridazine |
| Lamb J et al Science 2006 | 17008526 | control, Fulvestrant 10 nM, and Fulvestrant 1 uM |
| Lamb J et al Science 2006 | 17008526 | estradiol and control |
| Lamb J et al Science 2006 | 17008526 | genistein 1 uM and Genistein 10 uM |
| Lamb J et al Science 2006 | 17008526 | genistein vs. control |
| Lamb J et al Science 2006 | 17008526 | vorinostat vs. control |
| Li Z et al Cancer Cell 2007 | 18068631 | Eph EN tumor vs. Eph EN DN tumor |
| Li Z et al Cancer Cell 2007 | 18068631 | ETV6 NTRK3 transduced NIH 3T3 cells |
| Li Z et al Cancer Cell 2007 | 18068631 | WT mammary gland, FACS sorted tumor epithelial cells |
| Lin CY et al Breast Cancer Res | 17428314 | time series treatment by ER beta and E2 |
| 2007 | | |
| Loi S et al BMC Genomics 2008 | 18498629 | grade 1, 2, and 3 |
| using GPL570 | | |
| Loi S et al BMC Genomics 2008 | 18498629 | PR negative ER positive vs. PR positive ER positive |
| using GPL570 | | |
| Loi S et al BMC Genomics 2008 | 18498629 | tumor size comparison in GPL570 |
| using GPL570 | | |
| Loi S et al BMC Genomics 2008 | 18498629 | grade 1, 2, and 3 |
| using GPL96 | | |
| Loi S et al BMC Genomics 2008 | 18498629 | node negative vs. node positive |
| using GPL96 | | |
| Loi S et al BMC Genomics 2008 | 18498629 | RFS negative vs. RFS positive |
| using GPL96 | | |
| Loi S et al BMC Genomics 2008 | 18498629 | Tamoxifen treated vs. Untreated |
| using GPL96 | | |
| Loi S et al BMC Genomics 2008 | 18498629 | tumor size in GPL96 |
| using GPL96 | | |
| Loi S et al BMC Genomics 2008 | 18498629 | ER negative vs. ER positive |
| using GPL97 | | |
| Loi S et al BMC Genomics 2008 | 18498629 | grade 1, 2, and 3 |
| using GPL97 | | |
| Loi S et al BMC Genomics 2008 | 18498629 | node negative vs. node positive |
| using GPL97 | | |
| Loi S et al BMC Genomics 2008 | 18498629 | RFS negative vs. RFS positive |
| using GPL97 | | |
| Loi S et al BMC Genomics 2008 | 18498629 | tamoxifen untreated vs. tamoxifen treated |
| using GPL97 | | |
| Loi S et al BMC Genomics 2008 | 18498629 | ER negative vs. ER positive |
| using GPL96 | | |
| Lu X et al Breast Cancer Res Treat | 18297396 | breast cancer grades 1, 2, 3 |
| 2008 | | |
| Lu X et al Breast Cancer Res Treat | 18297396 | ductal breast cancer, lobular breast cancer, and mixed |
| 2008 | | |
| Lu X et al Breast Cancer Res Treat | 18297396 | ER negative vs. ER positive |
| 2008 | | |
| Lu X et al Breast Cancer Res Treat | 18297396 | HER2 negative vs. HER2 positive |
| 2008 | | |
| Lu X et al Breast Cancer Res Treat | 18297396 | LVI negative vs. LVI positive |
| 2008 | | |
| Lu X et al Breast Cancer Res Treat | 18297396 | node negative vs. node positive |
| 2008 | | |
| Lu X et al Breast Cancer Res Treat | 18297396 | tumor size comparisons |
| 2008 | | |
| Ma XJ et al Cancer Cell 2004 | 15193263 | grade 1, 2, and 3 |
| Ma XJ et al Cancer Cell 2004 | 15193263 | no recurrence vs. recurrence |
| Ma XJ et al Cancer Cell 2004 | 15193263 | PR negative vs. PR positive |
| Ma XJ et al PNAS 2003 | 12714683 | atypical ductal hyperplasia and ductal carcinoma |
| | | progression |
| Ma XJ et al PNAS 2003 | 12714683 | breast cancer grades I, II, III |
| Ma XJ et al PNAS 2003 | 12714683 | ER+ and ER− breast cancer |
| Ma XJ et al PNAS 2003 | 12714683 | Her2+ and Her2− breast cancer |
| Ma XJ et al PNAS 2003 | 12714683 | nod+ vs. nod− |
| Ma XJ et al PNAS 2003 | 12714683 | PR+ and PR− breast cancer |
| Miller LD et al PNAS 2005 using | 16141321 | breast cancer grade 1, 2, and 3 |
| GPL96 | | |
| Miller LD et al PNAS 2005 using | 16141321 | ER negative vs. ER positive |
| GPL96 | | |
| Miller LD et al PNAS 2005 using | 16141321 | node negative vs. node positive |
| GPL96 | | |
| Miller LD et al PNAS 2005 using | 16141321 | p53 wild type vs. p53 mutant |
| GPL96 | | |
| Miller LD et al PNAS 2005 using | 16141321 | PR negative vs. PR positive |
| GPL96 | | |
| Miller LD et al PNAS 2005 using | 16141321 | breast cancer grades 1, 2, and 3 |
| GPL97 | | |
| Miller LD et al PNAS 2005 using | 16141321 | ER negative vs. ER positive |
| GPL97 | | |
| Miller LD et al PNAS 2005 using | 16141321 | node negative vs. node positive |
| GPL97 | | |
| Miller LD et al PNAS 2005 using | 16141321 | p53 wild type vs. p53 mutant |
| GPL97 | | |
| Miller LD et al PNAS 2005 using | 16141321 | PR negative vs. PR positive |
| GPL97 | | |
| Oh DS et al J Clin Oncol 2006 using | 16505416 | 45 to 65 vs. more than 65 |
| GPL1390 | | |
| Oh DS et al J Clin Oncol 2006 using | 16505416 | any size and direct extension to chest wall or skin |
| GPL1390 | | |
| Oh DS et al J Clin Oncol 2006 using | 16505416 | grade 1, 2, and 3 |
| GPL1390 | | |
| Oh DS et al J Clin Oncol 2006 using | 16505416 | no relapse vs. relapse or die of disease |
| GPL1390 | | |
| Oh DS et al J Clin Oncol 2006 using | 16505416 | MCF7 Estrogen deprivation, transfected with GATA3 |
| GPL1708 | | |
| Oh DS et al J Clin Oncol 2006 using | 16505416 | any size and direct extension to chest wall or skin |
| GPL887 | | |
| Oh DS et al J Clin Oncol 2006 using | 16505416 | ER negative vs. ER positive |
| GPL887 | | |
| Oh DS et al J Clin Oncol 2006 using | 16505416 | grade 1, 2, and 3 |
| GPL887 | | |
| Oh DS et al J Clin Oncol 2006 using | 16505416 | no relapse vs. relapse or die of disease |
| GPL887 | | |
| Oh DS et al J Clin Oncol 2006 using | 16505416 | Node negative vs. Node positive |
| GPL887 | | |
| Oh DS et al J Clin Oncol 2006 using | 16505416 | ER negative vs. ER positive |
| GPL1390 | | |
| Oh DS et al J Clin Oncol 2006 using | 16505416 | ER negative vs. ER positive |
| GPL885 | | |
| Oh DS et al J Clin Oncol 2006 using | 16505416 | less than 45, 45 to 65, older than 65 |
| GPL887 | | |
| Perou CM et al PNAS 1999 | 10430922 | HMEC, abnormal HMEC, and breast cancer. |
| Ramaswamy S et al PNAS 2001 | 11742071 | breast cancer tissues vs. normal breast tissues |
| Smirnov DA et al Cancer Res 2006 | 16540638 | tumor tissues vs. normal tissues |
| Sotiriou C et al J Natl Cancer Inst | 16478745 | ER negative vs. ER positive |
| 2006 | | |
| Sotiriou C et al J Natl Cancer Inst | 16478745 | grades 1, 2, and 3 |
| 2006 | | |
| Sotiriou C et al J Natl Cancer Inst | 16478745 | tamoxifen treated primary breast cancer |
| 2006 | | |
| Sotiriou C et al J Natl Cancer Inst | 16478745 | tumor size comparison |
| 2006 | | |
| Sotiriou C et al PNAS 2003 | 12917485 | basal-like 1, basal-like 2, and Her-2/neu |
| Sotiriou C et al PNAS 2003 | 12917485 | ER status |
| Sotiriou C et al PNAS 2003 | 12917485 | grade status (I and III) |
| Sotiriou C et al PNAS 2003 | 12917485 | luminal-like 1, luminal-like 2, and luminal-like 3 |
| Stitziel NO et al Cancer Res2004 | 15574777 | MCF-7 CYT fraction vs. MCF-7 MEM fraction |
| Troester MA et al BMC Cancer 2006 | 17150101 | sham treated p53 RNAi, Dox-treated p53 RNAi cell lines |
| using GPL885 | | |
| Turashvili G et al BMC Cancer 2007 | 17389037 | ductal invasive vs. lobular invasive breast carcinomas |
| van de Vijver MJ et al N Engl J Med | 12490681 | breast cancer metastasis |
| 2002 | | |
| van de Vijver MJ et al N Engl J Med | 12490681 | ER negative breast cancer vs. ERCpositive breast |
| 2002 | | cancer |
| van de Vijver MJ et al N Engl J Med | 12490681 | lymphnode status |
| 2002 | | |
| van't Veer LJ et al Nature 2002 | 11823860 | distant metastasis and BRCA1 germline mutations |
| Wang Y et al Lancet 2005 | 15721472 | breast cancers without relapse vs. Breast cancers with |
| | | relapse |
| Wang Y et al Lancet 2005 | 15721472 | ER positive breast cancers vs. ER negative breast |
| | | cancers |
| Weigelt B et al Cancer Res 2005 | 16230372 | breast cancer subtype comparisons |
| Weigelt B et al Cancer Res 2005 | 16230372 | breast cancer subtype Luminal and Normal Breast-like |
| Weigelt B et al Cancer Res 2005 | 16230372 | HER2+ subtype, Basal-like, Normal Breast-like |
| Weigelt B et al PNAS 2003 | 14665696 | ER-alpha-negative vs. ER-alpha-positive |
| West M et al PNAS 2001 | 11562467 | ER and lymph node status |
| West M et al PNAS 2001 | 11562467 | ER+ tumors vs. ER− tumors |
| White SL et al Br J Cancer 2004 | 14710226 | C3.6 (EGF), C3.6 (Hrgb1), HB4a (EGF), and HB4a |
| Yau C et al Breast Cancer Res 2007 | 17850661 | node negative vs. node positive |
| Yu K et al Clin Cancer Res 2006 | 16740749 | ER negative vs. ER positive |
| Yu K et al Clin Cancer Res 2006 | 16740749 | grades 1, 2, and 3 |
| Yu K et al Clin Cancer Res 2006 | 16740749 | node negative vs. node positive |
| Yu K et al Clin Cancer Res 2006 | 16740749 | PR negative vs. PR positive |
| Zhou Y et al BMC Cancer 2007 | 17407600 | no recurrence vs. recurrence |

TABLE S2
List of breast cancer meta-signatures

| Signatures | Source | Method | Role |
|---|---|---|---|
| BRmet50 | 11 signatures | clustering and assembling | value test |
| BRSig70 | BR70 dataset | supervised selection | positive control |
| BRSig76 | BR76 dataset | supervised selection | positive control |
| BRmet[−1042] | 10 signatures | leave-one-out clustering | cross validation in BR1042 |
| BRmet[−1095] | 10 signatures | leave-one-out clustering | cross validation in BR1095 |
| BRmet[−1128] | 10 signatures | leave-one-out clustering | cross validation in BR1128 |
| BRmet[−1141] | 10 signatures | leave-one-out clustering | cross validation in BR1141 |
| BRmet[−1405] | 10 signatures | leave-one-out clustering | cross validation in BR1405 |
| BRmet[−1414] | 10 signatures | leave-one-out clustering | cross validation in BR1414 |
| BRmet[−1552] | 10 signatures | leave-one-out clustering | cross validation in BR1552 |
| BRmet[−2411] | 10 signatures | leave-one-out clustering | cross validation in BR2411 |
| BRmet[−544] | 10 signatures | leave-one-out clustering | cross validation in BR544 |

TABLE S3
Annotation of genes in BRmet50.

| Gene | Meta- | Expression | Role | Function |
|---|---|---|---|---|
| Symbol | Direction | in Cancer | in Cancer | Category |
| UBE2C | Up* | Up[31, 32] | Progression[31, 32] | Cell Cycle |
| KIF2C | Up | Up[33] | Progression[6] | Cell Cycle |
| TACC3 | Up | Up[34] | Progression[34] | Cell Cycle |
| MAD2L1 | Up | Up[35] | Progression[35] | Cell Cycle |
| AURKA | Up | Up[36] | Progression[36] | Cell Cycle |
| CEP55 | Up | Up[37] | Progression[38] | Cell Cycle |
| CCNB1 | Up | Up[39] | Progression[40] | Cell Cycle |
| RRM2 | Up | Up[41, 42] | Progression[17] | Cell Cycle |
| DLGAP5 | Up | Up[43] | Progression[43] | Cell Cycle |
| NEK2 | Up | Up[44] | Progression[45] | Cell Cycle |
| NDC80 | Up | Up[46] | NA* | Cell Cycle |
| UBE2S | Up | Up[11] | NA | Cell Cycle |
| CCNB2 | Up | Up[47] | NA | Cell Cycle |
| KIF20A | Up | Up[48] | NA | Cell Cycle |
| TRIP13 | Up | Up[49] | NA | Cell Cycle |
| CDKN3 | Up | Up[50] | NA | Cell Cycle |
| RAD51 | Up | Up[51] | Progression[51] | DNA Replication |
| KPNA2 | Up | Up[13] | Progression[13] | DNA Replication |
| TYMS | Up | Up[52] | NA | DNA Replication |
| CDT1 | Up | Up[53] | NA | DNA Replication |
| FEN1 | Up | Up[54] | NA | DNA Replication |
| RFC4 | Up | Up[55] | NA | DNA Replication |
| EZH2 | Up | Up[56] | Progression[56] | Proliferation |
| DDX39 | Up | Up[57] | Progression[57] | Proliferation |
| GTPBP4 | Up | Down[20] | Suppressor[20] | Proliferation |
| CCT5 | Up | Up[58] | NA | Protein Folding |
| HJURP | Up | NA | NA | Cell Cycle |
| SPAG5 | Up | NA | NA | Cell Cycle |
| KIF4A | Up | NA | NA | Cell Cycle |
| PRC1 | Up | NA | NA | Cell Cycle |
| KIF23 | Up | NA | NA | Cell Cycle |
| NUSAP1 | Up | NA | NA | Cell Cycle |
| CENPN | Up | NA | NA | Cell Cycle |
| LRP8 | Up | NA | NA | Cell Movement |
| GMPS | Up | NA | NA | DNA Replication |
| MCM10 | Up | NA | NA | DNA Replication |
| CDC45L | Up | NA | NA | DNA Replication |
| GARS | Up | NA | NA | Proliferation |
| C1orf106 | Up | NA | NA | NA |
| BTG2 | Down* | Down[21] | Suppressor[21] | Anti-Proliferation |
| SCUBE2 | Down | Down[22] | Suppressor[22] | Anti-Proliferation |
| OGN | Down | Down[59] | NA | Cellular |
| | | | | Assembly |
| SH3BGRL | Down | Down[60] | NA | Thioredoxin Fold |
| | | | | Proteins |
| COL14A1 | Down | NA | NA | Cellular |
| | | | | Assembly |
| SPARCL1 | Down | NA | NA | Cellular |
| | | | | Assembly |
| RAI2 | Down | NA | NA | Proliferation |
| KIF13B | Down | NA | NA | Cell Movement |
| QDPR | Down | NA | NA | Aamino Acid |
| | | | | and Oxidation |
| ALDH3A2 | Down | NA | NA | Lipid |
| | | | | Oxidoreductase |
| | | | | Activity |
| CIRBP | Down | NA | NA | mRNA |
| | | | | Stabilization |

*Up for up-regulation; Down for down-regulation; NA for not available. Meta-direction: concordant expression direction of the BRmet50 genes.

TABLE S4
Hazard ratio risks and log-rank tests in BR1141

| | BRmet50 Control | | BRsig70 | | BRsig76 | |
|---|---|---|---|---|---|---|
| Tumor features | HR (95% CI) | HR P | HR (95% CI) | HR P | HR (95% CI) | HR P |
| Tumor size | | | | | | |
| T1 | 2.5 (1.2-5.1) | 0.014 | 1.5 (0.6-3.7) | 0.386 | 1.0 (0.5-2.1) | 0.942 |
| T2 | 2.0 (1.2-3.3) | 0.009 | 1.8 (0.9-3.8) | 0.113 | 0.7 (0.4-1.2) | 0.209 |
| Lymph node | | | | | | |
| involvement | | | | | | |
| No | 2.2 (1.3-3.7) | 0.003 | 1.6 (0.8-3.0) | 0.193 | 0.8 (0.5-1.4) | 0.511 |
| Yes | 2.8 (1.4-5.6) | 0.004 | 2.8 (0.8-9.3) | 0.089 | 0.6 (0.3-1.4) | 0.245 |
| Tamoxifen | | | | | | |
| treatment | | | | | | |
| No | 2.7 (1.3-5.5) | 0.007 | 2.1 (1.0-4.6) | 0.063 | 1.1 (0.5-2.0) | 0.869 |
| Yes | 2.6 (1.5-4.6) | 0.001 | 1.7 (0.7-4.0) | 0.230 | 0.6 (0.3-1.0) | 0.041 |
| Differentiation | | | | | | |
| Good | 2.5 (0.8-7.4) | 0.105 | 2.4 (0.8-7.2) | 0.121 | 1.3 (0.4-3.8) | 0.682 |
| Intermediate | 2.9 (1.7-5.0) | <0.001 | 1.6 (0.8-3.4) | 0.219 | 0.7 (0.4-1.2) | 0.194 |
| Poor | 1.3 (0.5-3.1) | 0.602 | 0.2 (0-1.8) | 0.172 | 0.5 (0.2-1.1) | 0.086 |
| ER status | | | | | | |
| Negative | 1.9 (0.6-6.6) | 0.310 | 2.2 (0.3-16.3) | 0.456 | 0.9 (0.3-2.3) | 0.782 |
| Positive | 2.5 (1.6-4.0) | <0.001 | 1.8 (1.0-3.3) | 0.050 | 0.7 (0.4-1.1) | 0.103 |

TABLE S5
Comparison of signatures with common clinicopathologic factors by univariate hazard ratio model

| Classifiers | | BR1042 | BR1095 | BR1128 | BR1141 | GSE7390 |
|---|---|---|---|---|---|---|
| BRmet50* | HR (95% CI) | 2.8 (1.4-5.5) | 2.2 (1.4-3.3) | 2.8 (1.5-4.9) | 2.2 (1.5-3.3) | 1.7 (1.1-2.5) |
| | p-value | <0.01 | <0.01 | <0.01 | <0.01 | 0.03 |
| BRmet50 | HR (95% CI) | 2.0 (1.0-3.8) | 2.2 (1.4-3.3) | 2.8 (1.5-4.9) | 2.4 (1.6-3.6) | ND |
| control | p-value | 0.03 | <0.001 | <0.001 | <0.001 | ND |
| BRSig70 | HR (95% CI) | 2.0 (0.9-4.2) | 1.9 (1.3-3.0) | 1.9 (1.1-3.3) | 1.9 (1.1-3.4) | 1.1 (0.8-1.7) |
| | p-value | 0.07 | <0.01 | 0.01 | 0.03 | 0.52 |
| BRSig76 | HR (95% CI) | 1.1 (0.6-2.2) | 1.9 (1.2-3.0) | 2.0 (1.1-3.5) | 0.7 (0.5-1.1) | 1.4 (1.0-2.5) |
| | p-value | 0.7 | <0.01 | 0.02 | 0.16 | 0.06 |
| NPI** | HR (95% CI) | 1.6 (1.1-2.3) | 1.7 (1.3-2.1) | 2.2 (1.6-2.9) | 1.4 (1.1-1.8) | 1.1 (0.8-1.5) |
| | p-value | 0.02 | <0.01 | <0.01 | <0.01 | 0.46 |
| Size | HR (95% CI) | 1.6 (0.8-3.0) | 2.5 (1.6-3.7) | 3.2 (1.8-5.5) | 2.1 (1.4-3.3) | 1.2 (0.8-1.8) |
| | p-value | 0.15 | <0.01 | <0.01 | <0.01 | 0.36 |
| Grade | HR (95% CI) | 1.5 (1.0-2.3) | 1.8 (1.3-2.4) | 2.0 (1.4-3.0) | 1.3 (1.0-1.7) | 1.1 (0.8-1.4) |
| | p-value | 0.07 | <0.01 | <0.01 | 0.08 | 0.69 |
| Lymph | HR (95% CI) | 1.0 (1.0-1.0) | 2.2 (1.4-3.3) | 4.0 (2.3-6.9) | 1.5 (1.0-2.4) | 1.0 (1.0-1.0) |
| node | p-value | 1.00 | <0.01 | <0.01 | 0.05 | 1.00 |
| ER | HR (95% CI) | 0.7 (0.4-1.4) | 0.9 (0.5-1.6) | 1.3 (0.5-3.0) | 0.8 (0.5-1.3) | 0.8 (0.5-1.1) |
| | p-value | 0.36 | 0.76 | 0.59 | 0.31 | 0.22 |
| Age | HR (95% CI) | 1.0 (1.0-1.0) | 1.0 (1.0-1.0) | 1.0 (1.0-1.0) | 1.0 (1.0-1.0) | 1.0 (1.0-1.0) |
| | p-value | 0.45 | 0.72 | 0.73 | 0.89 | 0.42 |

*BRmet50 control signatures were tested by three BR datasets, and BRmet50 was examined in all breast cancer datasets.
**NPI: Nottingham Prognostic Index.

TABLE S6
Multivariate analysis of relapse risk among patients with breast cancer

| | BRmet50 | | | | | |
|---|---|---|---|---|---|---|
| | control | | BRSig70 | | BRSig76 | |
| | HR | | HR | | HR | |
| | (95% CI) | HR P | (95% CI) | HR P | (95% CI) | HR P |
| BR1042 | 2.3 | 0.04 | 1.7 (0.7-3.9) | 0.23 | 0.8 (0.4-1.7) | 0.54 |
| | (1.0-5.2) | | | | | |
| BR1095 | 1.8 | 0.02 | 1.5 (0.9-2.5) | 0.16 | 1.3 (0.8-2.2) | 0.26 |
| | (1.1-2.9) | | | | | |
| BR1128 | 2.0 | 0.03 | 1.4 (0.8-2.7) | 0.27 | 1.2 (0.6-2.3) | 0.49 |
| | (1.0-3.9) | | | | | |
| BR1141 | 2.5 | <0.01 | 1.6 (0.9-2.9) | 0.13 | 0.6 (0.4-1.0) | 0.05 |
| | (1.6-3.9) | | | | | |
| GSE7390 | ND | ND | 1.1 (0.6-2.0) | 0.76 | 2.0 (1.1-3.3) | 0.03 |

HR and HR P-values of signature effect adjusted by age, grade, tumor size, LN, ER, and NPI.
ND: not done.

TABLE S7
Summary of survival analysis p-values and c-indexes in breast cancer

| | BRmet50 | BRsig70 | BRsig76 | ONCO | TAMR13 | PAM50 | Genius | PIK3 | GGI |
|---|---|---|---|---|---|---|---|---|---|
| p-values | | | | | | | | | |
| METABRIC D | <0.001 | <0.001 | <0.001 | <0.001 | <0.001 | <0.001 | <0.001 | 0.001 | <0.001 |
| METABRIC V | <0.001 | <0.001 | <0.001 | <0.001 | <0.001 | <0.001 | <0.001 | 0.002 | <0.001 |
| GSE2607 | 0.004 | 0.005 | <0.001 | 0.01 | 0.814 | 0.078 | 0.814 | 0.814 | 0.814 |
| GSE7390 | 0.028 | 0.516 | 0.063 | 0.368 | 0.238 | 0.223 | 0.013 | 0.911 | 0.015 |
| GSE11121 | 0.027 | 0.012 | 0.183 | 0.002 | <0.001 | 0.004 | 0.003 | 0.012 | 0.122 |
| GSE17705 | 0.045 | 0.043 | 0.574 | 0.064 | 0.002 | 0.015 | 0.858 | 0.677 | 0.137 |
| GSE20624 | 0.001 | 0.037 | 0.037 | 0.003 | 0.037 | 0.037 | 0.037 | 0.037 | 0.037 |
| GSE20685 | <0.001 | 0.002 | <0.001 | <0.001 | 0.023 | 0.006 | 0.016 | 0.018 | 0.003 |
| GSE21653 | 0.014 | 0.121 | 0.396 | 0.001 | 0.007 | 0.123 | 0.027 | 0.11 | 0.06 |
| GSE25055 | <0.001 | <0.001 | <0.001 | <0.001 | 0.001 | <0.001 | <0.001 | <0.001 | <0.001 |
| GSE25065 | <0.001 | <0.001 | <0.001 | <0.001 | 0.89 | <0.001 | 0.597 | <0.001 | 0.082 |
| c-index | | | | | | | | | |
| METABRIC D | 0.6182 | 0.6125 | 0.5969 | 0.6379 | 0.5961 | 0.6159 | 0.6015 | 0.5726 | 0.6279 |
| METABRIC V | 0.6004 | 0.5905 | 0.5860 | 0.6142 | 0.5724 | 0.5838 | 0.5679 | 0.5638 | 0.6069 |
| GSE2607 | 0.5661 | 0.6498 | 0.5700 | 0.6712 | 0.5039 | 0.6342 | 0.5039 | 0.5039 | 0.5039 |
| GSE7390 | 0.5831 | 0.5469 | 0.5795 | 0.5524 | 0.5604 | 0.5578 | 0.5864 | 0.4745 | 0.5891 |
| GSE11121 | 0.6126 | 0.6199 | 0.5723 | 0.6353 | 0.6631 | 0.6359 | 0.6429 | 0.6135 | 0.586 |
| GSE17705 | 0.5845 | 0.569 | 0.5232 | 0.5724 | 0.6052 | 0.5865 | 0.5112 | 0.5185 | 0.5543 |
| GSE20624 | 0.5082 | 0.5063 | 0.5063 | 0.5989 | 0.5063 | 0.5063 | 0.5063 | 0.5063 | 0.5063 |
| GSE20685 | 0.6064 | 0.5978 | 0.6213 | 0.6006 | 0.5762 | 0.5885 | 0.5798 | 0.5839 | 0.5989 |
| GSE21653 | 0.5701 | 0.542 | 0.5209 | 0.6121 | 0.5866 | 0.5604 | 0.5644 | 0.568 | 0.5528 |
| GSE25055 | 0.6384 | 0.6524 | 0.6301 | 0.6440 | 0.6022 | 0.6604 | 0.6126 | 0.6830 | 0.6472 |
| GSE25065 | 0.646 | 0.6567 | 0.653 | 0.6884 | 0.5158 | 0.6894 | 0.5358 | 0.667 | 0.5766 |

Note:
METABRIC D and METABRIC V are discovery and validation datasets from METABRIC study [61], and Other datasets represented by GSE ID are available from NCBI GEO database.
There are eight published signatures in the study including BRsig70 [62], BRsig76) [63], ONCO (Oncotype DX) [64,65], TAMR13 [4], PAM50 [66], Genius [67], PIK3(PIK3CAGS278) [68], and GGI [69].

What is claimed is:

1. A method for characterizing a cancer in a subject, comprising:
(a) providing a biological sample from the subject;
(b) determining expression levels of genes in the biological sample to obtain a gene expression profile for the subject, wherein the genes comprise members of a meta-signature associated with the cancer; and
(c) comparing the profile of the subject to a reference, wherein the cancer is characterized based on a measurable difference in the expression levels of genes in the biological sample as compared to the reference.

2. The method of claim 1, wherein the characterizing comprises providing a diagnosis, prognosis and/or theranosis of the cancer.

3. The method of claim 2, including applying an algorithm for predicting a clinical outcome indicator from the gene expression profile of the subject, including genes comprising members of the meta-signature.

4. A method for evaluating treatment efficacy and/or progression of a cancer in a subject, comprising:
(a) providing a biological sample from the subject;
(b) determining expression levels of genes in the biological sample to obtain a gene expression profile for the subject, wherein the genes comprise members of a meta-signature associated with the cancer; and
(c) comparing the profile of the subject to a reference, wherein the treatment efficacy and/or progression of the cancer is evaluated based on a measurable difference in the expression levels of genes in the biological sample as compared to the reference.

5. The method of claim 4, and further comprising providing multiple biological samples from the subject collected at different time points, and determining expression levels of genes in each biological sample.

6. The method of claim 1, wherein the cancer is selected from a breast cancer, a lung cancer, a prostate cancer, and a colon cancer.

7. The method of claim 1, wherein the providing a biological sample from the subject comprises extracting mRNA from the biological sample and/or synthesizing cDNA.

8. The method of claim 1, wherein determining the expression levels of the genes in the biological sample includes sequencing the mRNA and/or DNA sequences of the biomarkers.

9. The method of claim 1, wherein the meta-signature includes 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 genes.

10. The method of claim 1, wherein the meta-signature is BRmet50.

11. A kit comprising a reagent to carry out the method of any of claim 1.

12. The kit of claim 10, comprising: one or more probes for determining the expression levels of the genes in the meta-signature.

13. The kit of claim 10, and further comprising reference data for one or more clinicopathologic features.
